(19) United States
(12) Patent Application Publication
(10) Pub. No.: US 2009/0223893 A1
(43) Pub. Date: Sep. 10, 2009

(54) Method and Device for Desalting an Analyte

(76) Inventors: Douglas T. Gjerde, Saratoga, CA (US); Lee Hoang, San Jose, CA (US); Chris Suh, San Jose, CA (US); Mark W. Abel, San Jose, CA (US)

Correspondence Address: PHYNEXUS, INC., 3670 CHARTER PARK DRIVE, SAN JOSE, CA, 95136, US

(21) Appl. No.: 12/435,381

(22) Filed: May 4, 2009

Related U.S. Application Data
(63) Continuation-in-part of application No. 11/292,707, filed on Dec. 1, 2005.

(51) Int. Cl. B01D 15/08

(52) U.S. Cl. 210/635

(57) ABSTRACT

The invention provides gel filtration columns for the purification of an analyte (e.g., a biological macromolecule, such as a peptide, protein or nucleic acid) from a sample solution, as well as methods for making and using such columns. The columns typically include a bed of gel filtration media positioned above a bottom frit or between a bottom and top frit. In some embodiments, the columns employ modified pipette tips as column bodies. In some embodiments, the invention provides methods and devices for desalting and/or buffer exchange of a sample.

### CROSS REFERENCE TO RELATED APPLICATIONS

This application is a continuation-in-part (CIP) of U.S. patent application Ser. No. 11/292,707 filed Nov. 30, 2005 the disclosure of which is incorporated herein by reference in its entirety for all purposes.

### FIELD OF THE INVENTION

This invention relates to methods and devices for separating or treating an analyte from a sample solution. The analytes can include biomolecules, particularly biological macromolecules such as proteins and peptides. The device and method of this invention are particularly useful in proteomics for size exclusion chromatography, gel filtration, buffer exchange, and desalting sample preparation and analysis with analytical technologies employing biochips, mass spectrometry and other instrumentation or biological analysis methods.

### BACKGROUND OF THE INVENTION

Size exclusion chromatography, Gel filtration chromatography, desalting or buffer exchange is a powerful technology for separating or treating analytes, including biomolecules. For example, it is one of the primary tools used for preparing protein samples prior to analysis by any of a variety of analytical techniques, including capillary electrophoresis, HPLC, mass spectrometry, surface plasmon resonance, nuclear magnetic resonance, x-ray crystallography, and the like, or biological assays including enzyme analysis, cell based assays or similar tests. With these techniques, typically only a small volume of sample is required. However, it is often critical that interfering contaminants be removed from the sample and that the analyte of interest is present at some minimum concentration. Thus, sample preparation methods are needed that permit the separation or treatment of small volume samples with minimal sample loss.

For the purpose of this invention size exclusion chromatography, gel filtration chromatography, desalting and buffer exchange are considered to be equivalent. This invention relies on making and using columns filled with water or buffer swollen media that have a small bed volume, and low cross sectional areas that are configured into a 96 well rack or plate format with 9.0 mm center-to-center format. The columns have very small bed volumes and small cross sectional areas and therefore can only use very small aliquots of liquid applied to the columns without overloading the column capacity with a particular sample, analyte or matrix component. Since the columns must fit into a 9.0 mm format the largest outside diameter a column in contact with an adjacent column is 9.0 mm. The low cross-sectional areas and small liquid aliquots used with these columns exhibit high resistance to liquid flow compared to the forces produced by the gravity of small aliquots to liquid to flow through the columns. Yet these columns are used of under gravity conditions to force sample and eluent through the column and to collect small aliquots of 30, 20 and even 10 uL. Furthermore, the columns have even flow so that they can be used with parallel up to 96 at a time in a single rack with the flow variation from column-to-column no greater than 50% relative of the fastest flowing column to the slowest flowing column.

The subject invention involves methods and devices for separating or treating an analyte from a sample solution using a packed bed of gel filtration media, e.g., a bed of water swollen gel-type beads. These methods, and the related devices and reagents, will be of particular interest to the life scientist, since they provide a powerful technology for treating biomolecules and other analytes of interest. However, the methods, devices and reagents are not limited to use in the biological sciences, and can find wide application in a variety of preparative and analytical contexts.

### SUMMARY OF THE INVENTION

The invention provides separation columns, many of which are characterized by the use of relatively small beds of extraction media.

In one embodiment, the instant invention provides an extraction column comprising: a column body having an open upper end, an open lower end, and an open channel between the upper and lower end of the column body; a bottom frit bonded to and extending across the open channel; a top frit bonded to and extending across the open channel between the bottom frit and the open upper end of the column body, the top frit having a low pore volume, wherein the top frit, bottom frit, and column body define an extraction media chamber; and a bed of extraction media positioned inside the extraction media chamber, said bed of extraction media having a volume of less than about 100 μL.

In some embodiments, the bed of extraction media comprises a packed bed of resin beads. Non-limiting examples of resin beads include water swollen gel resins.

In certain embodiments of the invention, the column comprises a packed bed of gel resin beads, e.g., agarose- or sepharose-based resins, polyacrylamide, dextran, and other hydrophilic polymer materials.

In certain embodiments of the invention, the bed of extraction media has a volume of between about 20 μL and 4000 μL, between about 100 μL and 2000 μL, or between about 200 μL and 1000 μL.

In certain embodiments of the invention, the bottom frit and/or the top frit is/are less than 200 microns thick.

In certain embodiments of the invention, the bottom frit and/or the top frit has/have a pore volume of 5 microliters or less.

In certain embodiments of the invention, the bottom frit and/or the top frit is/are a membrane screen, e.g., a nylon or polyester woven membrane.

In certain embodiments of the invention, the column body comprises a polycarbonate, polypropylene or polyethylene material.

In certain embodiments of the invention the column is configured into a plate or rack of columns with suitable 9.0 mm center-to-center column configuration to be used in a robotic liquid handler.

In certain embodiments of the invention, the column body comprises a luer adapter, a syringe, cylinder or a pipette tip.

In certain embodiments of the invention, the column comprises a lower tubular member comprising: the lower end of the column body, a first engaging end, and a lower open channel between the lower end of the column body and the first engaging end; and an upper tubular member comprising the upper end of the column body, a second engaging end, and an upper open channel between the upper end of the column body and the second engaging end, the top membrane screen of the extraction column bonded to and extending across the upper open channel at the second engaging end; wherein the first engaging end engages the second engaging end to form a sealing engagement. In some of these embodiments, the first engaging end has an inner diameter that matches the external diameter of the second engaging end, and wherein the first engaging end receives the second engaging end in a telescoping relation. The first engaging end optionally has a tapered bore that matches a tapered external surface of the second engaging end.

The invention further provides a method for separating an analyte from a sample solution comprising the steps of introducing a sample solution containing an analyte into the packed bed of gel filtration media packed into the bed of a desalting column of the invention, wherein the gel filtration media comprises a water swollen or buffer swollen matrix having pores either larger or smaller than the analyte, whereby the analyte either enters the pores or is excluded from the pores of the gel filtration media; introducing a chaser solvent into the bed of gel filtration media, whereby at least some fraction of the analyte is eluted from the gel filtration media and collected into a capture well, plate or rack of vials.

The invention further provides a method for separating an analyte from a sample solution comprising the steps of introducing a sample solution containing an analyte into the packed bed of gel filtration media of a desalting column of the invention, wherein the gel filtration media comprises an water swollen or buffer swollen matrix having pores larger than the analyte, whereby the analyte enters or partially enters the pores of the gel filtration media and other matrix materi are excluded or partially excluded from the pores of the gel filtration media and discarded; introducing a chaser solvent aliquot or series of aliquots into the bed of gel filtration media, whereby at least some fraction of the analyte is eluted from the gel filtration media and collected into a capture well, plate or rack of vials and separated from other sample matrix components.

The invention further provides a method for separating an analyte from a sample solution comprising the steps of introducing a sample solution containing an analyte into the packed bed of gel filtration media of a desalting column of the invention, wherein the gel filtration media comprises an water swollen or buffer swollen matrix having pores smaller than analyte, whereby the analyte is excluded or partially excluded the pores of the gel filtration media and other matrix materials enter or partially enter the pores of the gel filtration media; introducing a chaser solvent aliquot or series of aliquots into the bed of gel filtration media, whereby at least some fraction of the analyte is eluted from the gel filtration media an collected into a capture well, plate or rack of vials and separated from the other sample matrix components.

The invention further provides a method for desalting or buffer exchanging an analyte from a sample solution comprising the steps of introducing a sample solution containing an analyte into the packed bed of gel filtration media of a desalting column of the invention, wherein the gel filtration media comprises an water swollen or buffer swollen matrix having pores smaller than analyte but large enough for buffer or salts to enter, whereby the analyte is excluded or partially excluded the pores of the gel filtration media and other matrix salts enter or partially enter the pores of the gel filtration media; introducing a chaser solvent aliquot or series of aliquots into the bed of gel filtration media, whereby at least some fraction of the analyte is eluted from the gel filtration media and collected into a capture well, plate or rack of vials and is desalted and/or contains a new buffer and is separated from the original sample matrix salt or buffer.

The invention further provides a multiplexing of columns of limited cross sectional area that can fit into a configuration of 9.0 mm center-to-center spacing. The column may be any shape but can be configured into a 96 well format of 8 rows columns on one side and 12 rows of columns on the other size.

In certain embodiments of the method, the desalting column or columns moved are moved individually or in a rack into various stations in the robotic liquid handler.

In certain embodiments of the method, aliquots of liquid are applied to the top of the desalting columns with a pipette in a liquid handler.

In certain embodiments of the method, the top frit has properties that allow liquid to flow through the frit and into the column, but does not allow air to flow into the column thereby stopping the flow of liquid until the next aliquot of liquid is added to the top of the column.

### BRIEF DESCRIPTION OF THE FIGURES

FIG. 1 depicts an embodiment of the invention where the desalting column body is constructed from a tapered pipette tip.

FIG. 2 is an enlarged view of the extraction column of FIG. 1.

FIG. 3 depicts an embodiment of the invention where the desalting column is constructed from two cylindrical members.

FIGS. 4-8 show successive stages in the construction of the embodiment depicted in FIGS. 1 and 2.

FIG. 9 depicts an embodiment of the invention where the desalting column can take the form of a pipette tip.

FIG. 10 depicts a preferred embodiment of the general embodiment depicted in FIG. 9.

FIG. 11 depicts a pipette tip desalting column attached to an apparatus for determining column back pressure.

FIGS. 12 and 13 depict a method for determining the back pressure of a membrane frit as described in Example 2.

FIG. 14 depicts a porous frit, the back pressure of which is to be determined as described in Example 2.

FIG. 15 depicts a method of desalting a sample containing a protein analyte of interest.

FIG. 16 depicts an example of a gel filtration desalting columns with a collection plate and transfer tips.

FIG. 17A depicts a top view of a rack or plate for holding gel filtration columns. FIG. 17B depicts a cut-away view of a rack or plate.

FIG. 18 depicts the deck layout for a Beckman Biomek robotic liquid handler system.

FIG. 19 depicts the deck layout for a PhyNexus, Inc. MEA robotic liquid handler instrument.

### DESCRIPTION OF SPECIFIC EMBODIMENTS OF THE INVENTION

This invention relates to methods and devices for separating, desalting or buffer exchanging an analyte from a sample solution using a gravity flow column. The column contains gel filtration media co. The analytes can include biomolecules, particularly biological macromolecules such as proteins and peptides, polynucleotides, lipids and polysaccharides. The device and method of this invention are particularly useful in proteomics for sample preparation and analysis with analytical technologies employing biochips, mass spectrometry and other instrumentation and other technologies. The separation process generally results in the purification, desalting or buffer exchange of an analyte or analytes of interest.

In U.S. patent application Ser. No. 10/620,155, incorporated by reference herein in its entirety, methods and devices for performing low dead column extractions are described. The instant specification, inter alia, expands upon the concepts described in that application.

Gel filtration chromatography is a chromatographic method in which particles are separated based on their size or hydrodynamic volume. The method is [1] It is usually applied to large molecules such as proteins and other biomolecules such as polysaccharides and nucleic acids. Biologists and biochemists typically use a gel medium or packing material usually polyacrylamide, dextran or agarose. The advantages of this method include good separation of large molecules from the small molecules with a minimal volume of eluent and that various buffers can be used with affecting the separation process all while preserving the biological activity of the analyte particles.

The underlying principle of gel filtration chromatography is that particles of different sizes will elute or travel through a stationary phase at different rates resulting in the separation of a solution of particles based on size. Provided that all analyte particles are loaded simultaneously or near simultaneously, particles of the same size should elute together. Each size exclusion column has a range of molecular weights that can be separated. The exclusion limit defines the molecular weight at the upper end of this range and is where molecules are too large to be trapped in the stationary phase. The permeation limit defines the molecular weight at the lower end of the range of separation and is where molecules of a small enough size can penetrate into the pores of the stationary phase completely and all molecules below this molecular mass are so small that they elute as a single band.

Increasing the column length will enhance the resolution power of the column but will also increase column back pressure making gravity flow more difficult. Increasing the column diameter increases the capacity of the column but in this invention the diameter is limited by the configuration of the 96 well plate and rack. Proper column packing is important to maximize resolution: over-packed columns can collapse the pores in the beads, resulting in a loss of resolution and high and variable column backpressure. An under-packed column can improve the column backpressure but can reduce the relative surface area of the stationary phase accessible to smaller species, resulting in those species spending less time trapped in pores. Unlike affinity chromatography techniques, a solvent head at the top of the column can drastically diminish resolution as the sample diffuses prior to loading, broadening the downstream elution. The void volume is the total space surrounding the gel particles in a packed column.

In gravity columns the eluent is collected in volume aliquots known as fractions. In order to successfully operate the columns in parallel, the analytes must travel down the column in parallel at more or less the same time.

Other prior art gravity gel filtration desalting columns have larger cross sectional areas and therefore have lower backpressures.

Before describing the present invention in detail, it is to be understood that this invention is not limited to specific embodiments described herein. It is also to be understood that the terminology used herein for the purpose of describing particular embodiments is not intended to be limiting. As used in this specification and the appended claims, the singular forms “a”, “an” and “the” include plural referents unless the context clearly dictates otherwise. Thus, for example, reference to polymer bearing a protected carbonyl would include a polymer bearing two or more protected carbonyls, and the like.

Unless defined otherwise, all technical and scientific terms used herein have the same meaning as commonly understood by one of ordinary skill in the art to which the invention pertains. Although any methods and materials similar or equivalent to those described herein can be used in the practice or testing of the present invention, specific examples of appropriate materials and methods are described herein.

### DEFINITIONS

In describing and claiming the present invention, the following terminology will be used in accordance with the definitions set out below.

The term “bed volume” as used herein is defined as the volume of a bed of extraction media in an extraction column. Depending on how densely the bed is packed, the volume of the extraction media in the column bed is typically about one third to two thirds of the total bed volume; well packed beds have less space between the beads and hence generally have lower interstitial volumes.

The term “interstitial volume” of the bed refers to the volume of the bed of extraction media that is accessible to solvent, e.g., aqueous sample solutions, wash solutions and desorption solvents. For example, in the case where the extraction media is a chromatography bead (e.g., agarose or sepharose), the interstitial volume of the bed constitutes the solvent accessible volume between the beads, as well as any solvent accessible internal regions of the bead, e.g., solvent accessible pores. The interstitial volume of the bed represents the minimum volume of liquid required to saturate the column bed.

The term “dead volume” as used herein with respect to a column is defined as the interstitial volume of the extraction bed, tubes, membrane or frits, and passageways in a column. Some preferred embodiments of the invention involve the use of low dead volume columns, as described in more detail in U.S. patent application Ser. No. 10/620,155.

The term “elution volume” as used herein is defined as the volume of desorption or elution liquid added to the top of the column and into which the analytes are eluted and collected. The terms “desorption solvent,” “elution liquid” “chaser” liquid aliquot and the like are used interchangeably herein.

The terms “gel filtration column” and “gel filtration tip” and “rack of gel filtration columns” as used herein are defined as a column device used in gravity flow used in combination with robotic liquid handler containing a bed of solid phase gel filtration material, i.e., gel filtration media.

The term “frit” as used herein is defined as porous material for holding the gel filtration media in place in a column. A gel filtration media chamber is typically defined by a top and bottom frit positioned in an extraction column. In preferred embodiments of the invention the frit is a thin, low pore volume filter, e.g., a membrane screen. In some embodiments, the top frit is absent and the gel filtration media is positioned above the bottom frit.

The term “lower column body” as used herein is defined as the column bed and bottom membrane screen of a column.

The term “membrane screen” as used herein is defined as a woven or non-woven fabric or screen for holding the column packing in place in the column bed, the membranes having a low dead volume. The membranes are of sufficient strength to withstand packing and use of the column bed and of sufficient porosity to allow passage of liquids through the column bed. The membrane is thin enough so that it can be sealed around the perimeter or circumference of the membrane screen so that the liquids flow through the screen.

The term “sample volume”, as used herein is defined as the volume of the liquid of the original sample solution from which the analytes are separated or purified.

The term “upper column body”, as used herein is defined as the chamber and top membrane screen of a column.

The term “biomolecule” as used herein refers to biomolecule derived from a biological system. The term includes biological macromolecules, such as a proteins, peptides, polysaccharides, and nucleic acids.

The term “protein chip” is defined as a small plate or surface upon which an array of separated, discrete protein samples are to be deposited or have been deposited. These protein samples are typically small and are sometimes referred to as “dots.” In general, a chip bearing an array of discrete proteins is designed to be contacted with a sample having one or more biomolecules which may or may not have the capability of binding to the surface of one or more of the dots, and the occurrence or absence of such binding on each dot is subsequently determined. A reference that describes the general types and functions of protein chips is Gavin MacBeath, Nature Genetics Supplement, 32:526 (2002).

### Gel Filtration Desalting Columns

In accordance with the present invention there may be employed conventional chemistry, biological and analytical techniques within the skill of the art. Such techniques are explained fully in the literature. See, e.g. Chromatography, 5^{th} edition, PART A: FUNDAMENTALS AND TECHNIQUES, editor: E. Heftmann, Elsevier Science Publishing Company, New York (1992); ADVANCED CHROMATOGRAPHIC AND ELECTROMIGRATION METHODS IN BIOSCIENCES, editor: Z. Deyl, Elsevier Science BV, Amsterdam, The Netherlands, (1998); CHROMATOGRAPHY TODAY, Colin F. Poole and Salwa K. Poole, and Elsevier Science Publishing Company, New York, (1991).

In some embodiments of the subject invention the packed bed of gel filtration desalting media is contained in a column, e.g., a low dead volume column. Non-limiting examples of suitable columns, particularly low dead volume columns, are presented herein. It is to be understood that the subject invention is not to be construed as limited to the use of extraction beds in low dead volume columns, or in columns in general. For example, the invention is equally applicable to use with a packed bed of gel filtration desalting media as a component of a multi-well plate.

### Column Body

The column body is a tube having two open ends connected by an open channel, sometimes referred to as a through passageway. The tube can be in any shape, including but not limited to cylindrical or frustoconical, and of any dimensions consistent with the function of the column as described herein. In some preferred embodiments of the invention the column body takes the form of a pipette tip, a syringe, a luer adapter or similar tubular bodies. In embodiments where the column body is a pipette tip, the end of the tip wherein the bed of extraction media is placed can take any of a number of geometries, e.g., it can be tapered or cylindrical. In some case a cylindrical channel of relatively constant radius can be preferable to a tapered tip, for a variety of reason, e.g., solution flows through the bed at a uniform rate, rather than varying as a function of a variable channel diameter.

In some embodiments, one of the open ends of the column, sometimes referred to herein as the open upper end of the column, is adapted for attachment to a pump, either directly or indirectly. In some embodiments of the present invention, fluid enters the column through one end and exits through the other. In some embodiments, the invention provides extraction methods that involve a hybrid approach; that is, one or more fluids enter the column through one end and exit through the other, and one more fluids enter and exit the column through the same open end of the column, e.g., the lower end. Thus, for example, in some methods the sample solution and/or chaser solutions are introduced through the top of the column and exit through the bottom end.

The column body can be composed of any material that is sufficiently non-porous that it can retain fluid and that is compatible with the solutions, media, pumps and analytes used. A material should be employed that does not substantially react with substances it will contact during use of the extraction column, e.g., the sample solutions, the analyte of interest, the extraction media and desorption solvent. A wide range of suitable materials are available and known to one of skill in the art, and the choice is one of design. Various plastics make ideal column body materials, but other materials such as glass, ceramics or metals could be used in some embodiments of the invention. Some examples of preferred materials include polysulfone, polypropylene, polyethylene, polyethylene terephthalate, polyethersulfone, polytetrafluoroethylene, cellulose acetate, cellulose acetate butyrate, acrylonitrile PVC copolymer, polystyrene, polystyrene/acrylonitrile copolymer, polyvinylidene fluoride, glass, metal, silica, and combinations of the above listed materials.

### Gel Filtration Desalting Media

The extraction media used in the column is preferably a form of water-insoluble particle. Typically the analyte of interest is a protein, peptide or nucleic acid. The term “analyte” can refer to any compound of interest, e.g., to be analyzed or simply removed from a solution.

Many of the extraction media suitable for use in the invention are selected from a variety of classes of chromatography media. It has been found that many of these chromatography media and the associated chemistries are suited for use as solid phase gel filtration desalting media in the devices and methods of this invention.

Thus, examples of suitable extraction media include resin beads used for extraction and/or chromatography. Preferred resins include gel resins, pellicular resins, and macroporous resins.

The term “gel resin” refers to a resin comprising low-crosslinked bead materials that can swell in a solvent, e.g., upon hydration. Crosslinking refers to the physical linking of the polymer chains that form the beads. The physical linking is normally accomplished through a crosslinking monomer that contains bi-polymerizing functionality so that during the polymerization process, the molecule can be incorporated into two different polymer chains. The degree of crosslinking for a particular material can range from 0.1 to 30%, with 0.5 to 10% normally used. 1 to 5% crosslinking is most common. A lower degree of crosslinking renders the bead more permeable to solvent, thus making the functional sites within the bead more accessible to analyte. However, a low crosslinked bead can be deformed easily, and should only be used if the flow of eluent through the bed is slow enough or gentle enough to prevent closing the interstitial spaces between the beads, which could then lead to catastrophic collapse of the bed. Higher crosslinked materials swell less and may prevent access of the analytes and desorption materials to the interior functional groups within the bead. Generally, it is desirable to use as low a level of crosslinking as possible, so long is it is sufficient to withstand collapse of the bed. This means that in conventional gel-packed columns, slow flow rates may have to be used. In the present invention the back pressure is very low, and high liquid flow rates can be used without collapsing the bed. Surprisingly, using these high solvent velocities does not appear to reduce the capacity or usefulness of the bead materials. Common gel resins include agarose, sepharose, polystyrene, polyacrylate, cellulose and other substrates. Gel resins can be non-porous or micro-porous beads.

The low back pressure associated with certain columns of the invention results in some cases in the columns exhibiting characteristics not normally associated with conventional packed columns. For example, in some cases it has been observed that below a certain threshold pressure solvent does not flow through the column. This threshold pressure can be thought of as a “bubble point.” In conventional columns, the flow rate through the column generally increases from zero as a smooth function of the pressure at which the solvent is being pushed through the column. With many of the columns of the invention, a progressively increasing pressure will not result in any flow through the column until the threshold pressure is achieved. Once the threshold pressure is reached, the flow will start at a rate significantly greater than zero, i.e., there is no smooth increase in flow rate with pressure, but instead a sudden jump from zero to a relatively fast flow rate. Once the threshold pressure has been exceeded flow commences, the flow rate typically increases relatively smoothly with increasing pressure, as would be the case with conventional columns.

Soft gel resin beads, such as agarose and sepharose based beads, are found to work surprisingly well in columns and methods of this invention. In conventional chromatography fast flow rates can result in bead compression, which results in increased back pressure and adversely impacts the ability to use these gels with faster flow rates.

The average particle diameters of beads of the invention are typically in the range of about 1 μm to several millimeters, e.g., diameters in ranges having lower limits of 10 μm, 20 μm, 30 μm, 40 μm, 50 μm, 60 μm, 70 μm, 80 μm, 90 μm, 100 μm, 150 μm, 200 μm, 300 μm, or 500 μm, and upper limits of 50 μm, 60 μm, 70 μm, 80 μm, 90 μm, 100 μm, 150 μm, 200 μm, 300 μm, 500 μm.

The bead size that may be used depends somewhat on the bed volume and the cross sectional area of the column. A smaller bead will increase the column resolving power but will also increase the column backpressure.

### Frits

In some embodiments of the invention one or more frits is used to contain the bed of extraction in, for example, a column. Frits can take a variety of forms, and can be constructed from a variety of materials, e.g., glass, ceramic, metal, fiber. Some embodiments of the invention employ frits having a low pore volume, which contribute to reducing dead volume. The frits of the invention are porous, since it is necessary for fluid to be able to pass through the frit. The frit should have sufficient structural strength so that frit integrity can contain the extraction media in the column. It is desirable that the frit have little or no affinity for chemicals with which it will come into contact during the extraction process, particularly the analyte of interest. In many embodiments of the invention the analyte of interest is a biomolecule, particularly a biological macromolecule. Thus in many embodiments of the invention it desirable to use a frit that has a minimal tendency to bind or otherwise interact with biological macromolecules, particularly proteins, peptides and nucleic acids.

Frits of various pores sizes and pore densities may be used provided the free flow of liquid is possible and the beads are held in place within the extraction media bed.

In one embodiment, one frit (e.g., a lower frit) is bonded to and extends across the open channel of the column body. A second frit is bonded to and extends across the open channel between the bottom frit and the open upper end of the column body.

In this embodiment, the top frit, bottom frit and column body (i.e., the inner surface of the channel) define an extraction media chamber wherein a bed of extraction media is positioned. The frits should be securely attached to the column body and extend across the opening and/or open end so as to completely occlude the channel, thereby substantially confining the bed of extraction media inside the extraction media chamber. In preferred embodiments of the invention the bed of extraction media occupies at least 80% of the volume of the extraction media chamber, more preferably 90%, 95%, 99%, or substantially 100% of the volume. In some preferred embodiments the invention the space between the extraction media bed and the upper and lower frits is negligible, i.e., the frits are in substantial contact with upper and lower surfaces of the extraction media bed, holding a well-packed bed of extraction media securely in place.

In some preferred embodiments of the invention the bottom frit is located at the open lower end of the column body. This configuration is shown in the figures and exemplified in the Examples, but is not required, i.e., in some embodiments the bottom frit is located at some distance up the column body from the open lower end. However, in view of the advantage that comes with minimizing dead volume in the column, it is desirable that the lower frit and extraction media chamber be located at or near the lower end. In some cases this can mean that the bottom frit is attached to the face of the open lower end. However, in some cases there can be some portion of the lower end extending beyond the bottom frit. For the purposes of this invention, so long as the length of this extension is such that it does not substantially introduce dead volume into the extraction column or otherwise adversely impact the function of the column, the bottom frit is considered to be located at the lower end of the column body.

In some embodiments of the invention, the gel filtration desalting media chamber is positioned near one end of the column. The area of the column body channel above the extraction media chamber can be can be quite large in relation to the size of the extraction media chamber. The frits used in the invention are preferably characterized by having a low pore volume while still having low backpressure. Some preferred embodiments of the invention employ frits having pore volumes of less than 1 microliter (e.g., in the range of 0.015-1 microliter, 0.03-1 microliter, 0.1-1 microliter or 0.5-1 microliter), preferably less than 0.5 microliter (e.g., in the range of 0.015-0.5 microliter, 0.03-0.5 microliter or 0.1-0.5 microliter), less than 0.1 microliter (e.g., in the range of 0.015-0.1 microliter or 0.03-0.1 microliter) or less than 0.03 microliters (e.g., in the range of 0.015-0.03 microliter).

Frits of the invention preferably have pore openings or mesh openings of a size in the range of about 5-100 μm, more preferably 10-100 μm, and still more preferably 15-50 μm, e.g., about 43 μm. The performance of the column is typically enhanced by the use of frits having pore or mesh openings sufficiently large so as to minimize the resistance to flow. The use of membrane screens as described herein typically provide this low resistance to flow and hence better flow rates, reduced back pressure and minimal distortion of the bed of extraction media. The pore or mesh openings of course should not be so large that they are unable to adequately contain the extraction media in the chamber.

Some frits used in the practice of the invention are characterized by having a low pore volume relative to the interstitial volume of the bed of extraction media contained by the frit. Thus, in preferred embodiments of the invention the frit pore volume is equal to 10% or less of the interstitial volume of the bed of gel filtration desalting media (e.g., in the range 0.1-10%, 0.25-10%, 1-10% or 5-10% of the interstitial volume), more preferably 5% or less of the interstitial volume of the bed of extraction media (e.g., in the range 0.1-5%, 0.25-5% or 1-5% of the interstitial volume), and still more preferably 1% or less of the interstitial volume of the bed of extraction media (e.g., in the range 0.01-1%, 0.05-1% or 0.1-1% of the interstitial volume).

Some embodiments of the invention employ a thin frit, preferably less than 350 μm in thickness (e.g., in the range of 20-350 μm, 40-350 μm, or 50-350 μm), more preferably less than 200 μm in thickness (e.g., in the range of 20-200 μm, 40-200 μm, or 50-200 μm), more preferably less than 100 μm in thickness (e.g., in the range of 20-100 μm, 40-100 μm, or 50-100 μm), and most preferably less than 75 μm in thickness (e.g., in the range of 20-75 μm, 40-75 μm, or 50-75 μm).

Some preferred embodiments of the invention employ a membrane screen as the frit. The membrane screen should be strong enough to not only contain the extraction media in the column bed, but also to avoid becoming detached or punctured during the actual packing of the media into the column bed. Membranes can be fragile, and in some embodiments must be contained in a framework to maintain their integrity during use. However, it is desirable to use a membrane of sufficient strength such that it can be used without reliance on such a framework. The membrane screen should also be flexible so that it can conform to the column bed. This flexibility is advantageous in the packing process as it allows the membrane screen to conform to the bed of extraction media, resulting in a reduction in dead volume.

The membrane can be a woven or non-woven mesh of fibers that may be a mesh weave, a random orientated mat of fibers i.e. a “polymer paper,” a spun bonded mesh, an etched or “pore drilled” paper or membrane such as nuclear track etched membrane or an electrolytic mesh (see, e.g., U.S. Pat. No. 5,556,598). The membrane may be, e.g., polymer, glass, or metal provided the membrane is low dead volume, allows movement of the various sample and processing liquids through the column bed, may be attached to the column body, is strong enough to withstand the bed packing process, is strong enough to hold the column bed of beads, and does not interfere with the extraction process i.e. does not adsorb or denature the sample molecules.

The frit can be attached to the column body by any means which results in a stable attachment. For example, the screen can be bonded to the column body through welding or gluing. Gluing can be done with any suitable glue, e.g., silicone, cyanoacrylate glue, epoxy glue, and the like. The glue or weld joint must have the strength required to withstand the process of packing the bed of extraction media and to contain the extraction media with the chamber. For glue joints, glue should be employed that does not adsorb or denature the sample molecules.

For example, glue can be used to attach a membrane to the tip of a pipette tip-based extraction column, i.e., a column wherein the column body is a pipette tip. A suitable glue is applied to the end of the tip. In some cases, a rod may be inserted into the tip to prevent the glue from spreading beyond the face of the body. After the glue is applied, the tip is brought into contact with the membrane frit, thereby attaching the membrane to the tip. After attachment, the tip and membrane may be brought down against a hard flat surface and rubbed in a circular motion to ensure complete attachment of the membrane to the column body. After drying, the excess membrane may be trimmed from the column with a razor blade.

Alternatively, the column body can be welded to the membrane by melting the body into the membrane, or melting the membrane into the body, or both. In one method, a membrane is chosen such that its melting temperature is higher than the melting temperature of the body. The membrane is placed on a surface, and the body is brought down to the membrane and heated, whereby the face of the body will melt and weld the membrane to the body. The body may be heated by any of a variety of means, e.g., with a hot flat surface, hot air or ultrasonically. Immediately after welding, the weld may be cooled with air or other gas to improve the likelihood that the weld does not break apart.

Alternatively, a frit can be attached by means of an annular pip, as described in U.S. Pat. No. 5,833,927. This mode of attachment is particularly suited to embodiment where the frit is a membrane screen.

The frits of the invention, e.g., a membrane screen, can be made from any material that has the required physical properties as described herein. Examples of suitable materials include nylon, polyester, polyamide, polycarbonate, cellulose, polyethylene, nitrocellulose, cellulose acetate, polyvinylidine difluoride, polytetrafluoroethylene (PTFE), polypropylene, polysulfone, metal and glass. A specific example of a membrane screen is the 43 μm pore size Spectra/Mesh® polyester mesh material which is available from Spectrum Labs (Ranch Dominguez, Calif., Part Number 145837).

Pore size characteristics of membrane filters can be determined, for example, by use of method #F316-30, published by ASTM International, entitled “Standard Test Methods for Pore Size Characteristics of Membrane Filters by Bubble Point and Mean Flow Pore Test.”

The polarity of the membrane screen can be important. A hydrophilic screen will promote contact with the bed and promote the air-liquid interface setting up a surface tension. A hydrophobic screen would not promote this surface tension and therefore the threshold pressures to flow would be different. A hydrophilic screen is preferred in certain embodiments of the invention.

### Extraction Column Assembly

The extraction columns of the invention can be constructed by a variety of methods using the teaching supplied herein. In some preferred embodiments the extraction column can be constructed by the engagement (i.e., attachment) of upper and lower tubular members (i.e., column bodies) that combine to form the extraction column. Examples of this mode of column construction are described in the Examples and depicted in the figures.

In some preferred embodiments of the invention, an extraction column is constructed by the engaging outer and inner column bodies, where each column body has two open ends (e.g., an open upper end and an open lower end) and an open channel connecting the two open ends (e.g., a tubular body, such as a pipette tip). The outer column body has a first frit (preferably a membrane frit) bonded to and extending across the open lower end, either at the very tip of the open end or near the open end. The section of the open channel between the open upper end and the first frit defines an outer column body. The inner column body likewise has a frit (preferably a membrane frit) bonded to and extending across its open lower end.

To construct a column according to this embodiment, an extraction media of interest is disposed within the lower column body, e.g., by orienting the lower column body such that the open lower end is down and filling or partially filling the open channel with the resin, e.g., in the form of a slurry. The inner column body, or at least some portion of the inner column body, is then inserted into the outer column body such that the open lower end of the inner body (where the second frit is attached) enters the outer column body first. The inner column body is sealingly positioned within the open channel of the outer column body, i.e., the outer surface of the inner column body forms a seal with the surface of the open. The section of the open channel between the first and second frits contains the extraction media, and this space defines a media chamber. In general, it is advantageous that the volume of the media chamber (and the volume of the bed of extraction media positioned within said media chamber) is less than the outer column body, since this difference in volume facilitates the introduction of extraction media into the outer column body and hence simplifies the production process. This is particularly advantageous in embodiments of the invention wherein the extraction columns are mass produced.

In certain embodiments of the above manufacturing process, the inner column body is stably affixed to the outer column body by frictional engagement with the surface of the open channel.

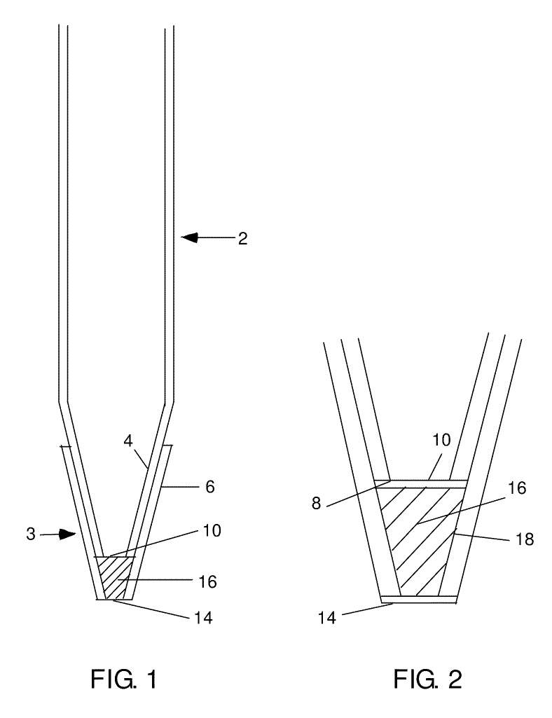

In some embodiments, one or both of the column bodies are tubular members, particularly pipette tips, sections of pipette tips or modified forms of pipette tips. For example, an embodiment of the invention wherein in the two tubular members are sections of pipette tips is depicted in FIG. 1 (FIG. 2 is an enlarged view of the open lower end and extraction media chamber of the column). This embodiment is constructed from a frustoconical upper tubular member 2 and a frustoconical lower tubular member 3 engaged therewith. The engaging end 6 of the lower tubular member has a tapered bore that matches the tapered external surface of the engaging end 4 of the upper tubular member, the engaging end of the lower tubular member receiving the engaging end of the upper tubular member in a telescoping relation. The tapered bore engages the tapered external surface snugly so as to form a good seal in the assembled column.

A membrane screen 10 is bonded to and extends across the tip of the engaging end of the upper tubular member and constitutes the upper frit of the extraction column. Another membrane screen 14 is bonded to and extends across the tip of the lower tubular member and constitutes the lower frit of the extraction column. The extraction media chamber 16 is defined by the membrane screens 10 and 14 and the channel surface 18, and is packed with extraction media.

The pore volume of the membrane screens 10 and 14 is low to minimize the dead volume of the column. The sample and desorption solution can pass directly from the vial or reservoir into the bed of extraction media. The low dead volume permits desorption of the analyte into the smallest possible desorption volume, thereby maximizing analyte concentration.

The volume of the extraction media chamber 16 is variable and can be adjusted by changing the depth to which the upper tubular member engaging end extends into the lower tubular member, as determined by the relative dimensions of the tapered bore and tapered external surface.

The sealing of the upper tubular member to the lower tubular in this embodiment is achieved by the friction of a press fit, but could alternatively be achieved by welding, gluing or similar sealing methods.

Note that in this and similar embodiments, a portion of the inner column body (in this case, a majority of the pipette tip 2) is not disposed within the first channel, but instead extends out of the outer column body. In this case, the open upper end of the inner column body is adapted for operable attachment to a pump, e.g., a pipettor.

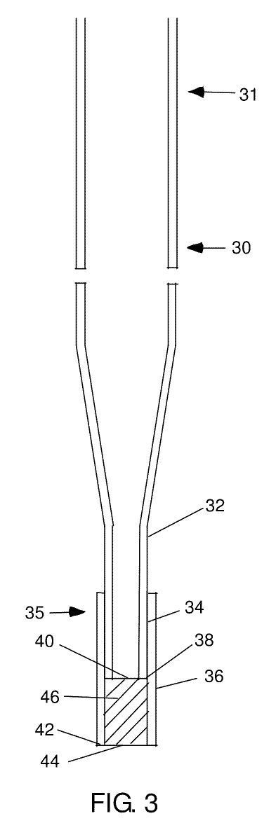

FIG. 3 depicts an embodiment of the invention comprising an upper and lower tubular member engaged in a telescoping relation that does not rely on a tapered fit. Instead, in this embodiment the engaging ends 34 and 35 are cylindrical, with the outside diameter of 34 matching the inside diameter of 35, so that the concentric engaging ends form a snug fit. The engaging ends are sealed through a press fit, welding, gluing or similar sealing methods. The volume of the extraction bed can be varied by changing how far the length of the engaging end 34 extends into engaging end 35. Note that the diameter of the upper tubular member 30 is variable; in this case it is wider at the upper open end 31 and tapers down to the narrower engaging end 34. This design allows for a larger volume in the column channel above the extraction media, thereby facilitating the processing of larger sample volumes and wash volumes. The size and shape of the upper open end can be adapted to conform to a pump used in connection with the column. For example, upper open end 31 can be tapered outward to form a better friction fit with a pump such as a pipettor or syringe.

A membrane screen 40 is bonded to and extends across the tip 38 of engaging end 34 and constitutes the upper frit of the extraction column. Another membrane screen 44 is bonded to and extends across the tip 42 of the lower tubular member 36 and constitutes the lower frit of the extraction column. The extraction media chamber 46 is defined by the membrane screens 40 and 44 and the open interior channel of lower tubular member 36, and is packed with extraction media.

In other embodiments of this general method of column manufacture, the entire inner column body is disposed within the first open channel. In these embodiments the first open upper end is normally adapted for operable attachment to a pump, e.g., the outer column body is a pipette tip and the pump is a pipettor. In some preferred embodiments, the outer diameter of the inner column body tapers towards its open lower end, and the open channel of the outer column body is tapered in the region where the inner column body frictionally engages the open channel, the tapers of the inner column body and open channel being complementary to one another. This complementarity of taper permits the two bodies to fit snuggly together and form a sealing attachment, such that the resulting column comprises a single open channel containing the bed of media bounded by the two frits.

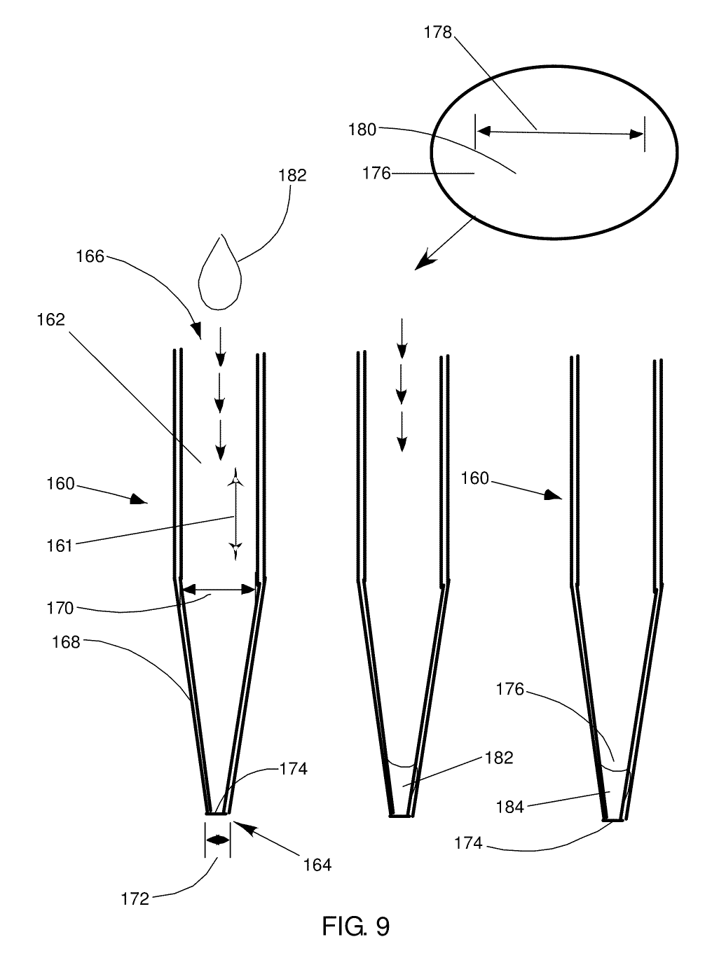

FIG. 9 illustrates the construction of an example of this embodiment of the extraction columns of the invention. This example includes an outer column body 160 having a longitudinal axis 161, a central through passageway 162 (i.e., an open channel), an open lower end 164 for the expulsion of fluid, and an open upper end 166. The outer column body includes a frustoconical section 168 of the through passageway 162, which is adjacent to the open lower end 164. The inner diameter of the frustoconical section decreases from a first inner diameter 170, at a position in the frustoconical section distal to the open lower end, to a second inner diameter 172 at the open lower end. A lower frit 174, preferably a membrane screen, is bonded to and extends across the open lower end 164. In a preferred embodiment a membrane frit can be bound to the outer column body by methods described herein, such as by gluing or welding. This embodiment further includes a ring 176 having an outer diameter 178 that is less than the first inner diameter 170 and greater than the second inner diameter 174. An upper frit 180, preferably a membrane screen, is bonded to and extends across the ring.

To construct the column, a desired quantity of extraction media 182, preferably in the form of a slurry, is introduced into the through passageway through the open upper end and positioned in the frustoconical section adjacent to the open lower end. The extraction media preferably forms a packed bed in contact with the lower frit 174. The ring 176 is then introduced into the through passageway through the open upper end and positioned at a point in the frustoconical section where the inner diameter of the frustoconical section matches the outer diameter 178 of the ring, such that the ring makes contact with and forms a seal with the surface of the through passageway. The upper frit, lower frit, and the surface of the through passageway bounded by the upper and lower frits define an extraction media chamber 184. The amount of media introduced into the column is normally selected such that the resulting packed bed substantially fills the extraction media chamber, preferably making contact with the upper and lower frits. That is, the bed is not tightly packed.

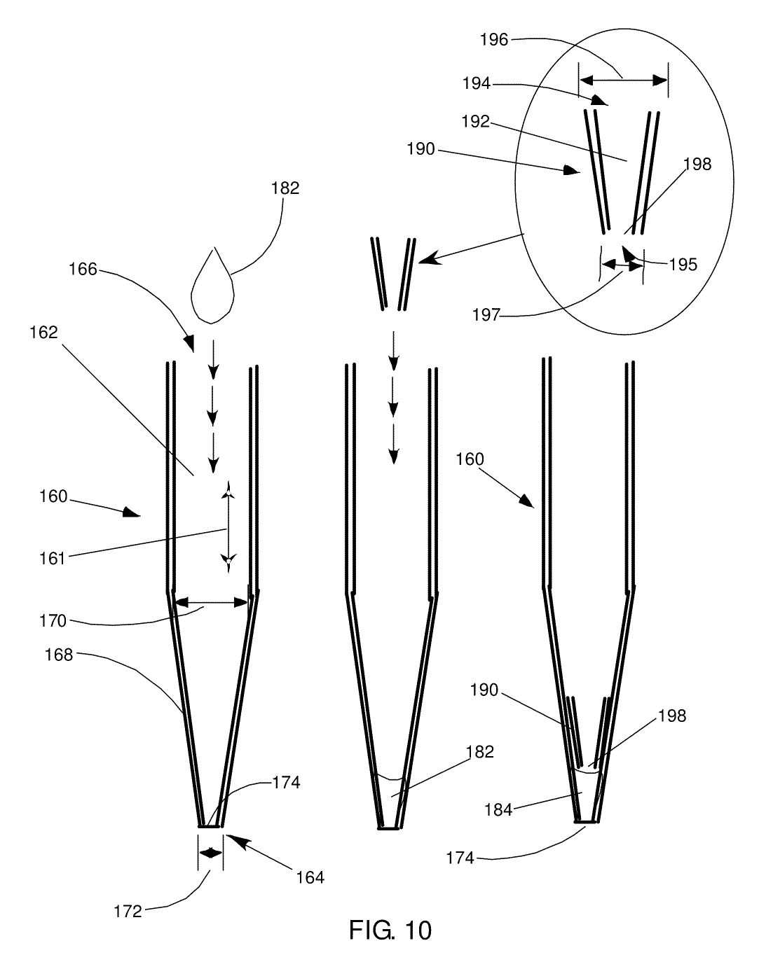

Note that the ring can take any of a number of geometries other than the simple ring depicted in FIG. 9, so long as the ring is shaped to conform to the internal geometry of the frustoconical section and includes a through passageway through which solution can pass. For example, FIG. 10 depicts a preferred embodiment wherein the ring takes the form of a frustoconical member 190 having a central through passageway 192 connecting an open upper end 194 and open lower end 195. The outer diameter of the frustoconical member decreases from a first outer diameter 196 at the open upper end to a second outer diameter 197 at the open lower end. The second outer diameter 197 is greater than the second inner diameter 172 and less than the first inner diameter 170. The first outer diameter 196 is less than or substantially equal to the first inner diameter 170. An upper frit 198 is bonded to and extends across the open lower end 195. The frustoconical member 190 is introduced into the through passageway of an outer column body containing a bed of media positioned at the lower frit 174. The tapered outer surface of the frustoconical member matches and the taper of the frustoconical section of the open passageway, and the two surfaces make a sealing contact. The extended frustoconical configuration of this embodiment of the ring facilitates the proper alignment and seating of the ring in the outer passageway.

Because of the friction fitting of the ring to the surface of the central through passageway, it is normally not necessary to use additional means to bond the upper frit to the column. If desired, one could use additional means of attachment, e.g., by bonding, gluing, welding, etc. In some embodiments, the inner surface of the frustoconical section and/or the ring is modified to improve the connection between the two elements, e.g., by including grooves, locking mechanisms, etc.

In the foregoing embodiments, the ring and latitudinal cross sections of the frustoconical section are illustrated as circular in geometry. Alternatively, other geometries could be employed, e.g., oval, polygonal or otherwise. Whatever the geometries, the ring and frustoconical shapes should match to the extent required to achieve an adequately sealing engagement. The frits are preferably, but not necessarily, positioned in a parallel orientation with respect to one another and perpendicular to the longitudinal axis.

Typically the analyte is a biomolecule, the solvent is an aqueous solution, typically containing a buffer, salt, and/or surfactants to solubilize and stabilize the biomolecule.

The back pressure of a column will depend on the average bead size, bead size distribution, average bed length, average cross sectional area of the bed, back pressure due to the frit and viscosity of flow rate of the liquid passing through the bed. For a 200 uL bed described in this application, the backpressure at 2 mL/min flow rate ranged from 0.5 to 5 psi. For a GE G-25 Sephadex column having bed size of 200 uL the range was 0.7 psi at a flow rate of 1 ml/min. Other column dimensions will result in backpressures ranging from, e.g., 0.1 psi to 30 psi depending on the parameters described above.

In some embodiments, the invention provides columns characterized by small bed volumes, small average cross-sectional areas, and/or low backpressures. This is in contrast to previously reported columns having small bed volumes but having higher backpressures, e.g., for use in HPLC. Examples include backpressures under normal operating conditions (e.g., 2 mL/min in a column with 200 μL bed) less than 30 psi, less than 10 psi, less than 5 psi, less than 2 psi, less than 1 psi, less than 0.5 psi, less than 0.1 psi, less than 0.05 psi, less than 0.01 psi. An advantage of low back pressures is that it allows gravity flow.

Because of the low backpressures, many of these columns can be run using only gravity to drive solution through the column. Other technologies having higher backpressures need a higher pressure to drive solution through, e.g., centrifugation at relatively high speed. This limits the use of these types of columns to resin beads that can withstand this pressure without collapsing.

The term “cross-sectional area” refers to the area of a cross section of the bed of extraction media, i.e., a planar section of the bed generally perpendicular to the flow of solution through the bed and parallel to the frits. In the case of a cylindrical or frustoconical bed, the cross section is generally circular and the cross sectional area is simply the area of the circle (area=pi×r²). In embodiments of the invention where the cross sectional area varies throughout the bed, such as the case in many of the preferred embodiments described herein having a tapered, frustoconical shape, the average cross-sectional area is an average of the cross sectional areas of the bed. As a good approximation, the average cross-sectional area of a frustoconical bed is the average of the circular cross-sections at each end of the bed. The average cross-sectional area of the bed of extraction media can be quite small in some of the columns of the invention, particularly low backpressure columns. Examples include cross-sectional areas of less than about 100 mm², less than about 50 mm², less than about 20 mm², less than about 10 mm², less than about 5 mm², or less than about 1 mm². Thus, some embodiments of the invention involve ranges of cross sectional areas extending from a lower limit of 0.1, 0.5, 1, 2, 3, 5, 10 or 20 mm² to an upper limit of 1, 2, 3, 5, 10, 20, 30, 40, 50, 60, 70, 80, 90 or 100 mm².

Often it is desirable to automate the method of the invention. For that purpose, the subject invention provides a device for performing the method comprising columns containing a packed bed of gel filtration desalting media, placed in a rack in a liquid handler.

The automated means for operating the liquid handler is controlled by software. This software controls the pipettes, and can be programmed to introduce desired liquids into to tops of the gel filtration column using pipette tips as well as to move the rack of columns from position to position to collect aliquots fractions of liquid.

For example, in certain embodiments the invention provides a general method for passing liquid through a rack of packed-bed pipette tip columns comprising the steps of:
a) providing a first a rack of columns comprising:
   i. a column body having an open upper end for communication with a pump, a first open lower end for the uptake and dispensing of fluid, and an open passageway between the upper and lower ends of the column body;
   ii. a bottom frit attached to and extending across the open passageway;
   iii. a top frit attached to and extending across the open passageway between the bottom frit and the open upper end of the column body, wherein the top frit, bottom frit, and surface of the passageway define a media chamber;
   iv. a first packed bed of media positioned inside the media chamber;
b) applying liquid aliquots to the top of the rack of columns using robotic liquid handlers and pipettes and liquid passing through the rack of columns by gravity flow
c) collecting liquid aliquots of liquid from the bottom of rack of columns in individual wells or vials.

In certain embodiments, the storage liquid is a water miscible solvent having a viscosity greater than that of water. In certain embodiments the water miscible solvent has a boiling point greater than 250° C. The water miscible solvent can comprise 50% of the storage liquid. In some preferred embodiments the water miscible solvent comprises a diol, triol, or polyethylene glycol of n=2 to n=150, e.g., glycerol.

The various embodiments described above that involve adjusting or controlling head pressure are particularly useful in embodiments of the invention that involve the use of automated or robotic liquid handling systems, e.g., automated multichannel pipettors. Thus, the various columns discussed can be different columns used simultaneously on a multichannel automated system, or in some cases different columns used sequentially on the same channel.

Packing the gel filtration desalting columns is performed in a manner that results in inform flow. Every column is different and one column cannot flow exactly same as the other column(s). A slurry of resin is introduced into the column and the resin is settled by pressure, vacuum or gravity. The slurry is made up of gel filtration desalting media that has been swollen overnight or in some cases few days in water or buffer. In some embodiments the slurry is made with water. In other embodiments the slurry is made with a high viscosity solvent to slow the settling of material to facilitate easier packing and more uniform bed volume of the slurry into the column. In other embodiments, the slurry is balanced with a salt or molecular species that makes a high density solvent. Non limiting examples of high density additives include cesium chloride, potassium carbonate, sucrose, glucose, glycerol and propylene glycol.

After the slurry is packed into the column the frit is placed on top of the bed. Compression of the bed is limited and at least uniform so that the column flow through the low bed volume, low diameter gravity column is uniform. In some embodiments a floating frit is used and then in some cases set into place with wall compression or welding. In other embodiments, the frit at the bottom of the insert is flexible so that when the top frit is positioned into place. Low pressure is exerted to the bed of the column and bed compression is limited. In some embodiments, the top frit is spongy and flexible so that when the frit is placed at top of the column the frit is compressed rather than the bed. In some embodiments, just the top of the column is used with no frit. In this case care must be taken not to disturb the resin bed when sample and chaser aliquots are added.

### Multiplexing

In some embodiments of the invention a plurality of columns is run in a parallel fashion, e.g., multiplexed. This allows for the simultaneous, parallel processing of multiple samples. A description of multiplexing of extraction capillaries is provided in U.S. patent application Ser. Nos. 10/434,713 and 10/733,534, and the same general approach can be applied to the columns and devices of the subject invention.

Multiplexing can be accomplished, for example, by arranging the columns in parallel so that fluid can be passed through them concurrently. Multiplexing is the heart of this invention. Due to the small size of the column, especially the cross sectional area, and the small liquid aliquots applied to the column at the various processing steps, it is difficult to achieve uniform flow through the columns. Uniform flow is achieved by using columns that are uniformly packed and have similar column backpressures, adding liquid uniformly to the top of each column just above the frit so that no air enters the column, using a top frit that stops the flow of liquid when the meniscus of liquid reaches the top of the column, and collecting drop of liquid flow evenly across the columns.

Even with these precautions the method usually has a pause built into the step so that the flow can catch up to the slowest column in the rack or plate. Examples of pause times include 0.5, 1, 2, 5, 10, 15, 17, 20, 25 and 30 minutes. After the pause time has elapsed, all the menisci have reached the top frit. If the top frit is absent, all the menisci have reached the top of the bed of media.

Generally, a certain specified amount of volume is processed or flowed through a column within a range of time even with some variations of the columns. These parameters include the frit backpressure, cross section area of the column, resin type and compressibility, resin average size, size distribution of the resin, compression of the resin within the column and finally the buffer or liquid that is flowing through the column. For example, 200 mL resin bed gel filtration columns of the invention packed with Sephadex G-25 fine resin can process 600 mL aliquot of water in 8-9 minutes and a 70 mL of water in 1.5-2.5 minutes. However, in another example with the same gel filtration column, using 6M guanidine (a dense buffer) slowed the flow rate or increased the processing time. In this example, to process 70 mL of the 6M guanidine buffer took between 3-5 minutes. A 20 mL aliquot can be processed as quickly as 1 minute and as slow as 5 minutes due to parameters listed above. For a 50 mL aliquot, the aliquot can be processed as quickly as 3 minutes and as slow as 15 minutes again due to the parameters listed above. For a given set of columns and conditions, the flow rates do not vary more than +/−20%, +/−10%, +/−5%, +/−2.5% of the average flow time within the set of columns.

In one embodiment, sample can be arrayed from an extraction column to a plurality of predetermined locations, for example locations on a chip or microwells in a multi-well plate. A precise liquid processing system can be used to dispense the desired volume of eluent at each location. For example, a transfer pipette containing 50 μL of sample or chaser buffer are dispensed into the rack or plate of gel filtration columns using a robotic system such as those commercially available from Zymark (e.g., the SciClone sample handler), Tecan (e.g., the Genesis NPS, Aquarius or TeMo) or Cartesian Dispensing (e.g., the Honeybee benchtop system), Packard (e.g., the MiniTrak5, Evolution, Platetrack. or Apricot), Beckman (e.g., the FX-96) and Matrix (e.g., the Plate Mate 2 or SerialMate). This can be used for high-throughput assays, crystallizations, etc.

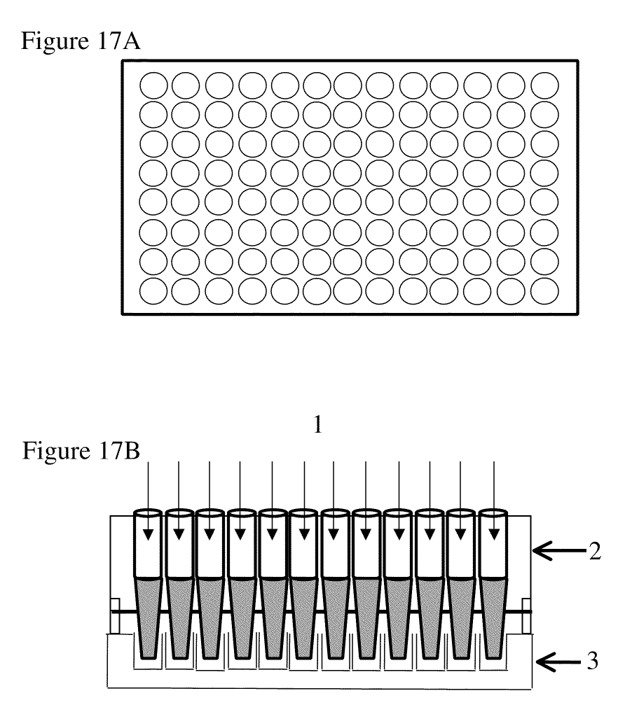

FIG. 16, 17A and 17B depict examples of a rack of columns used in a multiplexed extraction system. FIG. 16 shows eight gel filtration desalting columns with collection plate 4. The gel filtration columns can be packed with different types of gel filtration resins with varying resin bed sizes 5. The liquid/fluid chaser aliquots are added to upper end 1 of the columns by transfer tips 6 with liquid/fluid chaser aliquots and the liquid/fluid chaser aliquots are processed in one direction by gravity flow 2 (FIG. 17B). The flow of the liquid stops when liquid meniscus 7 (in FIG. 16) reaches the frit. The top frit screen prevents air from entering the resin bed so that column does not dry, crack or channel, which would result in poor performance. The method is paused long enough for the meniscus in each of the columns to reach the top frit. In some embodiments, the top frit is absent, in which case the method is paused long enough for the meniscus in each of the columns to reach the top of the bed. At this point, when liquid flow is stopped for all columns, the next aliquot of liquid is added.

FIG. 17A shows the top view of the 96 gel filtration columns in a rack and/or plate sitting on top of a collection plate. FIG. 17B shows the side view of 96 gel filtration columns in rack or plate 2 sitting on top of collection plate 3. 96 gel filtration columns are held in rack or plate 2. The rack/plate serve three purposes. First, it holds 96 gel filtration columns in standard 96-well format. Second, the PhyNexus rack or plate allows the robotic instrument to move 96 columns simultaneously from one position to another. Third, the PhyNexus rack or plate positions the end of the gel filtration columns close to the bottom of the collection plate. The plate is designed to collect all of the eluent that has passed through the column as the liquid/fluid chaser aliquots are added to the open upper end 1 of the columns and processed by gravity flow.

The robotic liquid handler systems include a controller for pipetting and positioning, columns, plates and racks. The controller is attached to a computer which can be programmed for pipetting control. The controller controls the timing and rate the plunger rack is moved, which in turn is used to control the flow of solution through the columns. The software allows control of the dispensing of aliquots to along with delays between operations.

In some embodiments, the invention provides a multiplexed extraction system comprising a plurality of extraction columns of the invention, e.g., gel filtration desalting columns having small beds of packed gel resins. The system can include a pipette, racks and columns in operative engagement with the columns, useful for allowing fluid through the columns in a multiplex fashion, i.e., concurrently. In some embodiments, each column is addressable. The term “addressable” refers to the ability to deliver the fluid individually to each column. An addressable column is one in which the flow of fluid through the column can be controlled independently from the flow through any other column which may be operated in parallel. For example, when pipette pumps are used, then separate transfer tips are used at each column. Because the columns are addressable, a controlled amount of liquid can be accurately manipulated in each column. Various embodiments of the invention can also include samples racks, instrumentation for controlling fluid aliquot manipulation, etc. The controller can be manually operated or operated by means of a computer. The computerized control is typically driven by the appropriate software, which can be programmable, e.g., by means of user-defined scripts.

The invention also provides software for implementing the methods of the invention. For example, the software can be programmed to control manipulation of solutions and addressing of columns into sample vials, collection vials, for spotting or introduction into some analytical device for further processing.

The invention also includes kits comprising one or more reagents and/or articles for use in a process relating to gel filtration, e.g., buffers, standards, solutions, columns, sample containers, etc.

### Recovery of Native Proteins

In some embodiments, the extraction devices and methods of the invention are used to purify proteins that are functional, active and/or in their native state, i.e., non-denatured. This is accomplished by performing the gel filtration desalting process under non-denaturing conditions. Non-denaturing conditions encompasses the entire protein separation process, General parameters that influence protein stability are well known in the art, and include temperature (usually lower temperatures are preferred), pH, ionic strength, the use of reducing agents, surfactants, elimination of protease activity, protection from physical shearing or disruption, radiation, etc. The particular conditions most suited for a particular protein, class of proteins, or protein-containing composition vary somewhat from protein to protein.

In one embodiment, the gel filtration desalting process is performed under conditions that do not irreversibly denature the protein. Thus, even if the protein is eluted in a denatured state, the protein can be renatured to recover native and/or functional protein. In this embodiment, the protein is adsorbed to the extraction surface under conditions that do not irreversibly denature the protein, and eluting the protein under conditions that do not irreversibly denature the protein. The conditions required to prevent irreversible denaturation are similar to those that are non-denaturing, but in some cases the requirements are not as stringent. For example, the presence of a denaturant such as urea, isothiocyanate or guanidinium chloride can cause reversible denaturation. The eluted protein is denatured, but native protein can be recovered using techniques known in the art, such as dialysis to remove denaturant. Likewise, certain pH conditions or ionic conditions can result in reversible denaturation, readily reversed by altering the pH or buffer composition of the eluted protein.

The recovery of non-denatured, native, functional and/or active protein is particularly useful as a preparative step for use in processes that require the protein to be non-denatured in order for the process to be successful. Non-limiting examples of such processes include analytical methods such as binding studies, activity assays, enzyme assays, X-ray crystallography and NMR.

### Method for Desalting a Sample

In some embodiments, the invention is used to change the composition of a solution in which an analyte is present. An example is the desalting of a sample, where some or substantially all of the salt (or other constituent) in a sample is removed or replaced by a different salt (or non-salt constituent). The removal of potentially interfering salt from a sample prior to analysis is important in a number of analytical techniques, e.g., mass spectroscopy. These processes will be generally referred to herein as “desalting,” with the understanding that the term can encompass any of a wide variety of processes involving alteration of the solvent or solution in which an analyte is present, e.g., buffer exchange or ion replacement.

Desalting and buffer exchange can be accomplished by means of a desalting tip column containing a packed bed of size exclusion media, e.g., a Sephadex G-10, G-15, G-25, G-50 or G-75 resin. Methodology for making and using size exclusion desalting tip columns is provided below in Example 3.

In some embodiments of the above-described procedure, the bed of desalting media is a size exclusion resin, such as Sephadex. This size exclusion media is typically hydrated by passing water or some aqueous solution, e.g., a buffer, through it. In some embodiments, the interstitial space of the bed is substantially full of water or aqueous solution, while in other embodiments liquid is blown out of the interstitial space prior to passing an analyte-containing sample through the bed.

The high molecular weight analyte is typically a high molecular weight biomolecule such as a protein. The low mass chemical entity is typically a salt, ion, or a non-charged low molecular weight molecule component of a buffer or other solution. As a result of passage through the desalting bed, the high molecular weight sample is separated from some, most, or substantially all of the low mass chemical entity, i.e., the sample is desalted. That is, prior to desalting, the sample solution contains high molecular weight analyte and low mass chemical entity at an initial concentration ratio (as calculated by dividing the concentration of high molecular weight analyte by the concentration of low mass chemical entity). After desalting, the product of the process contains either high molecular weight analyte, either substantially free of the low mass chemical entity, or, if there is some low mass chemical entity present, the final concentration ratio (as calculated by dividing the concentration of high molecular weight analyte by the concentration of low mass chemical entity in the eluted sample) is greater than the initial concentration ratio.

In some embodiments, the initial sample solution is eluted directly from a pipette tip column and into the bed of desalting media. This is an example of a stacking format, as exemplified in Example 3.

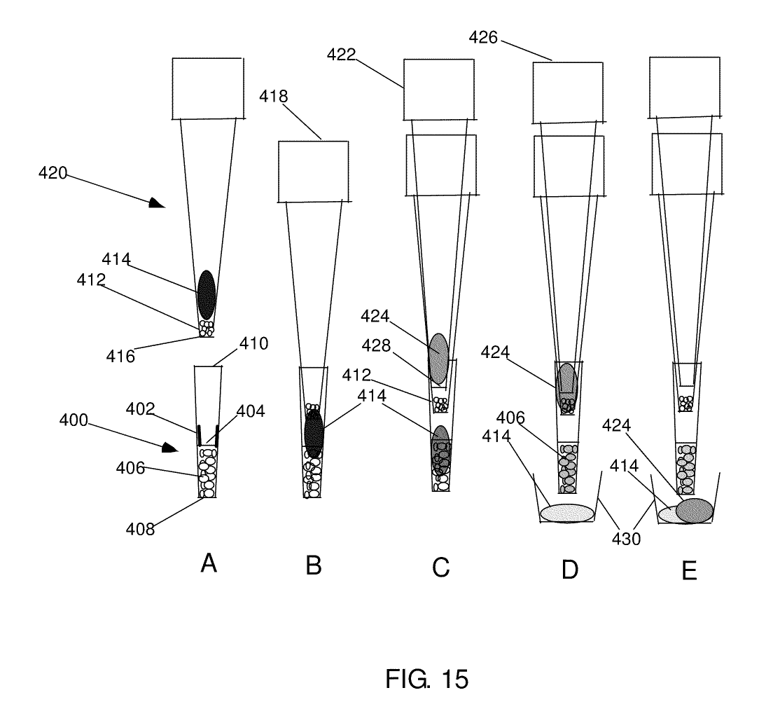

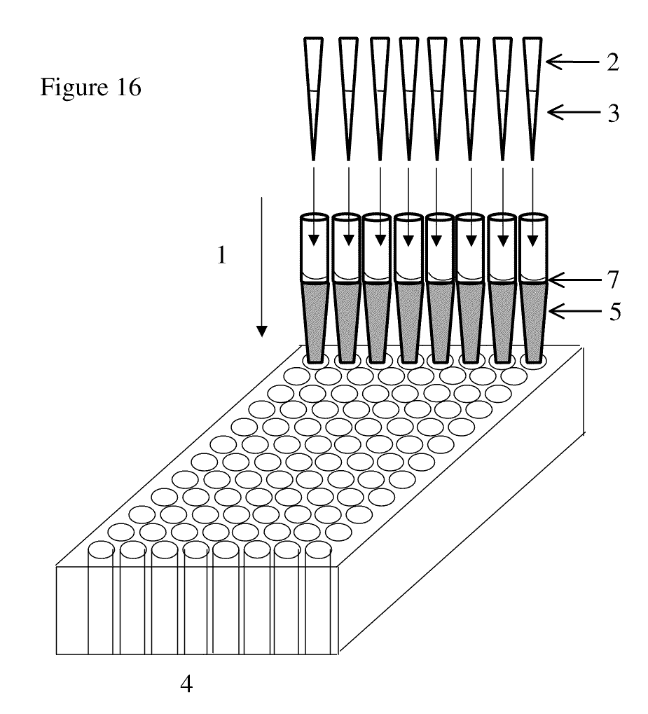

In some embodiments, the high molecular mass analyte is eluted by means of a chaser solution, as described in Example 3 and depicted in FIG. 15.

The uniformity of the PhyTip gel filtration columns is measured in terms of Coefficient of Variability (CV). The measurable parameters include volume collected, flow rate, mass of collected molecules, and concentration of molecules in collected volume. After addition of 5 μL to a PhyTip gel filtration column, the collected volume ranges between 4.25-5.75 μL with a CV of 15. Larger volumes will have lower CV values. For collecting volumes of 50 μL, the collected volume will range from 46-52 μL with a CV value of 6. In one embodiment, the CV is 10. In another embodiment, the CV is 20. For collecting 10, 20, 50, and 100 μL, the CV values range from 20 to 5.

The flow rate and collected volume is directly related to the mass and concentration of the target molecule(s) collected provided that the columns are manufactured appropriately. In one embodiment, loading 70 μL of a 2 mg/mL sample of human immunoglobulin G (140 μg total) results in collection of 120-140 μg, with a CV value of 8. In another embodiment, 20 μL of 2 mg/mL samples yields 30-40 μg with a CV value of 14. For dilute or small volume samples containing 5-900 ng, the CV value is 20. For samples containing 1 μg to 500 μg the CV values is 10. For concentrated samples of 600-1000 μg, the CV value is 15. In addition to the column performance, other factors influence the mass recovery. These factors include loss of sample due to too much dilution, or loss of sample due to too much mass, both situations will increase the CV values.

### Analytical Techniques

Extraction columns and associated methods of the invention find particular utility in preparing samples of analyte for analysis or detection by a variety of analytical techniques. In particular, the methods are useful for purifying an analyte, class of analytes, aggregate of analytes, etc, from a biological sample, e.g., a biomolecule originating in a biological fluid. It is particularly useful for use with techniques that require small volumes of pure, concentrated analyte. In many cases, the results of these forms of analysis are improved by increasing analyte concentration. In some embodiments of the invention the analyte of interest is a protein, and the extraction serves to purify and concentrate the protein prior to analysis. The methods are particularly suited for use with label-free detection methods or methods that require functional, native (i.e., non-denatured protein), but are generally useful for any protein or nucleic acid of interest.

These methods are particularly suited for application to proteomic studies, the study of protein-protein interactions, and the like. The elucidation of protein-protein interaction networks, preferably in conjunction with other types of data, allows assignment of cellular functions to novel proteins and derivation of new biological pathways. See e.g., Curr. Protein Pept. Sci. 2003 4(3):159-81.

Many of the current detection and analytical methodologies can be applied to very small sample volumes, but often require that the analyte be enriched and purified in order to achieve acceptable results. Conventional sample preparation technologies typically operate on a larger scale, resulting in waste because they produce more volume than is required. This is particularly a problem where the amount of starting sample is limited, as is the case with many biomolecules. These conventional methods are generally not suited for working with the small volumes required for these new methodologies. For example, the use of conventional packed bed chromatography techniques tend to require larger solvent volumes, and are not suited to working with such small sample volumes for a number of reasons, e.g., because of loss of sample in dead volumes, on frits, etc. See U.S. patent application Ser. No. 10/434,713 for a more in-depth discussion of problems associated with previous technologies in connection with the enrichment and purification of low abundance biomolecules.

Liquid flow is resisted by the backpressure of the column and by surface tension effects within the column, particularly in the bed and at the interface of the bed and frits. Surface tension can arise from the interaction of liquid with the packed bed of media and/or with the frit. This surface tension results in an initial resistance to flow of liquid through the bed of extraction media, described elsewhere herein as a form of “bubble point.” As a result, a certain minimum threshold of head pressure must be generated before liquid will commence flowing through the bed. In addition, there is the backpressure of the column that must be overcome in order for liquid to flow through the bed. Thus, in operation of the column a sufficiently negative head pressure must be generated to overcome backpressure and surface tension effects prior to flow commencing through the bed. The magnitude of the pressure drop across the column will to some extent depend upon the backpressure and surface tension, which in turn depends upon the size of the bed, the nature of the media, the nature of the packing, the nature of the frits, and the interaction of the frits with the bed.

During the course of using the columns of the invention, the pressure drop of any given column will vary during the course of the process. For example, let us consider an embodiment where multiple pipette tip columns and a programmable multi-channel pipettor are used.

The pressure drop present at any given step in the separation process will vary from column to column. This variation can be the result of any of a number of factors, including the slight variations from column to column, reflecting subtle difference in the packing of the bed and of the interaction of the bed with the frits and with the liquid, i.e., differential surface tension and back pressure effects.

This can be the case where multiple columns are run sequentially (in series). This can also be the case when multiple columns are run concurrently and/or in parallel, e.g., as accomplished via a multi-channel pipettor or robotic liquid handling system. Because of subtle differences from tip to tip, different head pressures can develop from tip to tip.

In certain embodiments, the invention provides methods of addressing the problems associated with the above-described variations in head pressure.

Maintaining Pipette Tip Columns and Polymer Beads in a Wet State

In certain embodiments, the invention provides methods of storing pipette tip columns in a wet state, i.e., with a “wet bed” of extraction media. This is useful in it allows for preparing the columns and then storing for extended periods prior to actual usage without the bed drying out, particularly where the extraction media is based on a resin, such as a gel resin. For example, it allows for the preparation of wet columns that can be packaged and shipped to the end user, and it allows the end user to store the columns for a period of time before usage. In many cases, if the bed were allowed to dry out it would adversely affect column function, or would require a time-consuming extra step of re-hydrating the column prior to use.

The maintenance of a wet state can be particularly critical wherein the bed volume of the packed bed is small, e.g., in a range having a lower limit of, 20 μL, or 40 μL, and an upper limit of 50 μL, 100 μL, 200 μL, 300 μL, 500 μL, 1 mL, 2 mL, 5 mL. Typical ranges would include 200 to 2000 μL

The wet tip results from producing a tip having a packed bed of media wherein a substantial amount of the interstitial space is occupied by a liquid. Substantial wetting would include beds wherein at least 25% of the interstitial space is occupied by liquid, and preferably at least 50%, 70%, 80%, 90%, 95%, 98%, 99%, or substantially the entire interstitial space is occupied by liquid. The liquid can be any liquid that is compatible with the media, i.e., it should not degrade or otherwise harm the media or adversely impact the packing. Preferably, it is compatible with purification and/or extraction processes intended to be implemented with the column. For example, trace amounts of the liquid or components of the liquid should not interfere with solid phase extraction chemistry if the column is intended for use in a solid phase extraction. Examples of suitable liquids include water, various aqueous solutions and buffers, and various polar and non-polar solvents described herein. The liquid might be present at the time the column is packed, e.g., a solvent in which the extraction media is made into a slurry, or it can be introduced into the bed subsequent to packing of the bed.

In certain preferred embodiments, the liquid is a solvent that is water miscible and that is relatively non-volatile and/or has a relatively high boiling point (and preferably has a relatively high viscosity relative to water). A “relatively high boiling point” is generally a boiling point greater than 100° C., and in some embodiments of the invention is a boiling point at room temperature in range having a lower limit of 100° C., 110° C., 120° C., 130° C., 140° C., 150° C., 160° C., 170° C., 180° C., 190° C., 200° C., or higher, and an upper limit of 150° C., 160° C., 170° C., 180° C., 190° C., 200° C., 220° C., 250° C., 300° C., or even higher. Illustrative examples would include alcohol hydrocarbons with a boiling point greater than 100° C., such as diols, triols, and polyethylene glycols (PEGs) of n=2 to n=150 (PEG-2 to PEG-150), PEG-2 to PEG-20, 1,3-butanediol and other glycols, particularly glycerol and ethylene glycol. The water miscible solvent typically constitutes a substantial component of the total liquid in the column, wherein “a substantial component” refers to at least 5%, and preferably at least 10%, 20%, 30%, 40%, 50%, 60%, 70%, 80%, 90%, 95%, 98%, 99%, or substantially the entire extent of the liquid in the column.

An advantage of these non-volatile solvents is that they are much less prone to evaporate than the typical aqueous solutions and solvents used in extraction processes. Thus, they will maintain the bed in a wet state for much longer than more volatile solvents. For example, an interstitial space filled with glycerol will in many cases stay wet for days without taking any additional measures to maintain wetness, while the same space filled with water would soon dry out. These solvents are particularly suitable for shipping and storage of gel type resin columns having agarose or sepharose beds. Other advantageous properties of many of these solvents, is that they are viscous so it is not easily displaced from column from shipping vibrations and movements, they are bacterial resistant, they do not appreciably penetrate or solvate agarose, sepharose, and other types of packing materials, and they stabilize proteins. Glycerol in particular is a solvent displaying these characteristics. Note that any of these solvents can be used neat or in combination with water or another solvent, e.g., pure glycerol can be used, or a mixture of glycerol and water or buffer, such as 50% glycerol or 75% glycerol.

One advantage of glycerol is that its presence in small quantities has negligible effects on many solid-phase extraction process. A tip column can be stored in glycerol to prevent drying, and then used in an extraction process without the need for an extra step of expelling the glycerol. Instead, a sample solution (typically a volume much greater than the bed volume, and hence much greater than the volume of glycerol) is loaded directly on the column by drawing it up through the bed and into the head space as described elsewhere herein. The glycerol is diluted by the large excess of sample solution, and then expelled from the column along with other unwanted contaminants during the loading and wash steps.

Note that relatively viscous, non-volatile solvents of the type described above, particularly glycerol and the like, are generally useful for storing polymer beads, especially the resin beads as described herein, e.g., agarose and sepharose beads and the like. Other examples of suitable beads would include xMAP® technology-based microspheres (Luminex, Inc., Austin, Tex.). Storage of polymer beads as a suspension in a solution comprising one or more of these solvents can be advantageous for a number of reasons, such as preventing the beads from drying out, reducing the tendency of the beads to aggregate, and inhibiting microbial growth. The solution can be neat solvent, e.g., 100% glycerol, or a mixture, such as an aqueous solution comprising some percentage of glycerol. The solution can also maintain the functionality of the resin bead by maintaining proper hydration, and protecting any affinity binding groups attached to the bead, particularly relatively fragile functional groups, such as certain biomolecules, e.g., proteins.

Factors that can affect the rate at which a column dries include the ambient temperature, the air pressure, and the humidity. Normally columns are stored and shipped at atmospheric pressure, so this is usually not a factor that can be adjusted. However, it is advisable to store the columns at lower temperatures and higher humidity in order to slow the drying process. Typically the columns are stored under room temperature conditions. Room temperature is normally about 25° C., e.g., between about 20° C. and 30° C. In some cases it is preferable to store the pipette tip columns at a relatively low temperature, e.g., between about 0° C. and 30° C., between 0° C. and 25° C., between 0° C. and 20° C., between 0° C. and 15° C., between 0° C. and 10° C., or between 0° C. and 4° C. In many cases tips of the invention may be stored at even lower temperatures, particularly if the tip is packed with a liquid having a lower freezing point than water, e.g., glycerol.

In one embodiment, the invention provides a pipette tip column that comprises a bed of media, the interstitial space of which has been substantially full of liquid for at least 24 hours, for at least 48 hours, for at least 5 days, for at least 30 days, for at least 60 days, for at least 90 days, for at least 6 months, or for at least one year. “Substantially full of liquid” refers to at least 25%, 50%, 70%, 80%, 90%, 95%, 98%, 99%, or substantially the entire interstitial space being occupied by liquid, without any additional liquid being added to the column over the entire period of time. For example, this would include a column that has been packaged and shipped and stored for a substantial amount of time after production.

In one embodiment, the invention provides a packaged pipette tip column packaged in a container that is substantially full of liquid, wherein the container maintains the liquid in the pipette tip to the extent that less than of 10% of the liquid is (or will be) lost when the tip is stored under these conditions for at least 24 hours, for at least 48 hours, for at least 5 days, for at least 30 days, for at least 60 days, for at least 90 days, for at least 6 months, or for at least one year.

In another embodiment, the invention provides a pipette tip column that comprises a bed of media, the interstitial space of which is substantially full of liquid, wherein the liquid is escaping (e.g., by evaporation or draining) at a rate such that less than 10% of the liquid will be lost when the column is stored at room temperature for 24 hours, 48 hours, 5 days, 30 days, 60 days, 90 days, six months or even one year.

In many cases, the wet pipette tip columns described above (e.g., the column that has been wet for an extended period of time and/or the column that is losing liquid only at a very slow rate) is packaged, e.g., in a pipette tip rack. The rack is a convenient means for dispensing the pipette tip columns, and for shipping and storing them as well. Any of a variety of formats can be used; racks holding 96 tips are common and can be used in conjunction with multi-well plates, multi-channel pipettors, and robotic liquid handling systems.

All publications and patent applications mentioned in this specification are herein incorporated by reference to the same extent as if each individual publication or patent application was specifically and individually indicated to be incorporated by reference.

Having now generally described the invention, the same will be more readily understood through reference to the following examples, which are provided by way of illustration, and are not intended to be limiting of the present invention, unless so specified.

### EXAMPLES

The following preparations and examples are given to enable those skilled in the art to more clearly understand and practice the present invention. They should not be construed as limiting the scope of the invention, but merely as being illustrative and representative thereof.

### Example 1

### Preparation of a Gel Filtration Desalting Column Body from Pipette Tips

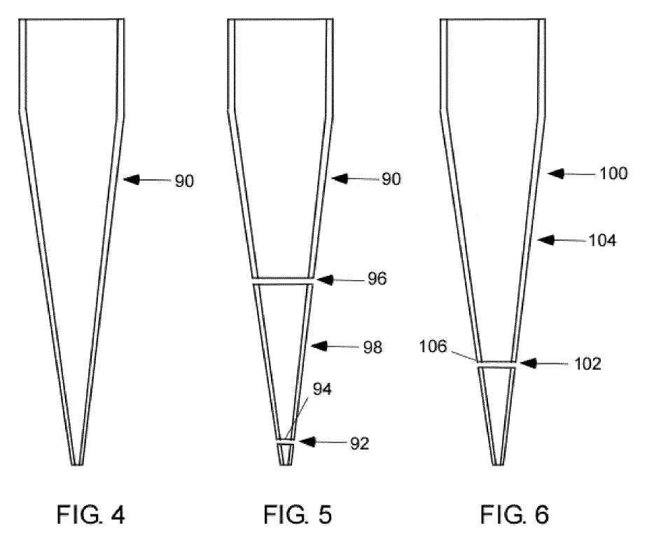

Two 1000 μL polypropylene pipette tips of the design shown in FIG. 4 (VWR, Brisbane, Calif., PN 53508-987) were used to construct one extraction column. In this example, two columns were constructed: a 10 μL bed volume and 20 μL bed volume. To construct a column, various components were made by inserting the tips into several custom aluminum cutting tools and cutting the excess material extending out of the tool with a razor blade to give specified column lengths and diameters.

Referring to FIG. 5, the first cut 92 was made to the tip of a pipette tube 90 to form a 1.25 mm inside diameter hole 94 on the lower column body, and a second cut 96 was made to form a lower column body segment 98 having a length of 15.0 mm.

Referring to FIG. 6, a cut 102 was made to the separate pipette tip 100 to form the upper column body 104. To make a 10 μL bed volume column, the cut 102 was made to provide a tip 106 outside diameter of 2.09 mm so that when the upper body 104 was inserted into the lower body 98, the column height of the solid phase media bed 114 (FIG. 8) was 4.5 mm. To make a 20 μL bed volume column, the cut 102 was made to provide a tip outside diameter of 2.55 mm cut so that when the upper body was inserted into the lower body, the column height of the solid phase media bed 114 (FIG. 8) was 7.0 mm.

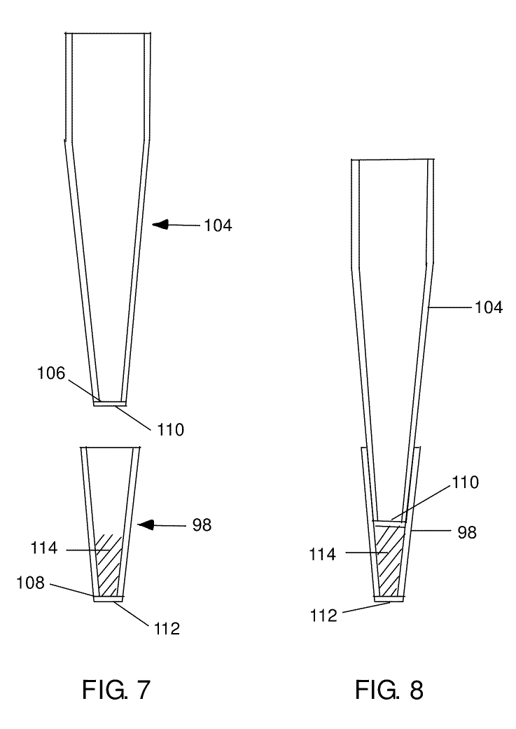

Referring to FIG. 7, a 43 μm pore size Spectra/Mesh® polyester mesh material (Spectrum Labs, Ranch Dominguez, Calif., PN 145837) was cut into discs by a circular cutting tool (Pace Punches, Inc., Irvine, Calif.) and attached to the ends 106 and 108 of the upper column and lower column bodies to form the membrane screens 110 and 112. The membrane screens were attached using PLASTIX® cyanoacrylate glue (Loctite, Inc., Avon, Ohio). The glue was applied to the polypropylene body and then pressed onto the membrane screen material. Using a razor blade, excess mesh material was removed around the outside perimeter of each column body end.

Referring to FIG. 8, the upper column body 104 is inserted into the top of the lower column body segment 98 and pressed downward to compact the solid phase media bed 114 to eliminate excess dead volume above the top of the bed.

### Example 2

### Comparison of Frit Backpressures

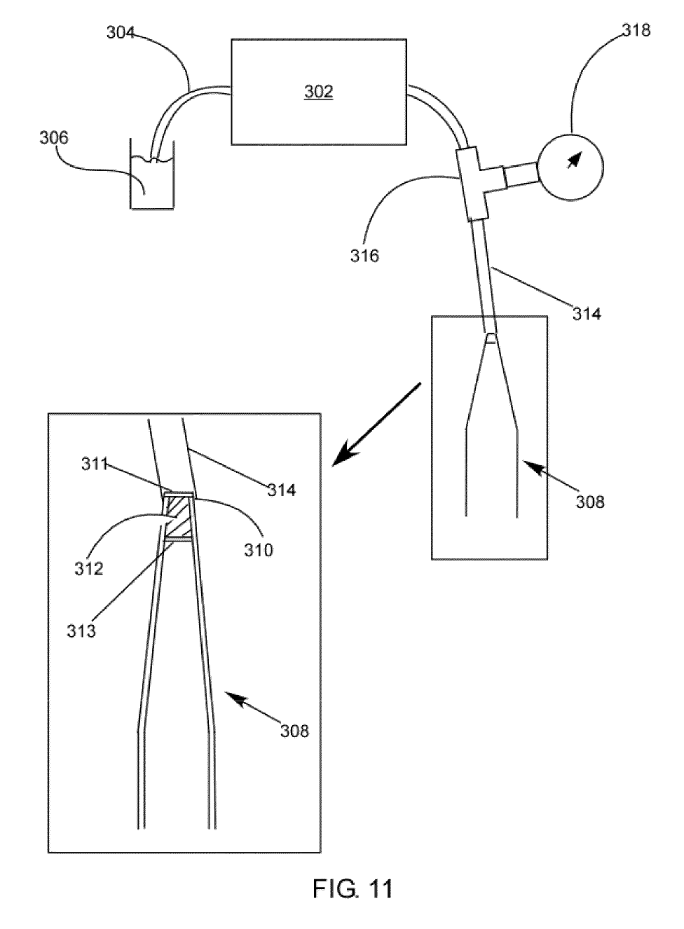

The backpressure was determined for a number of screen frits and porous polymer frits using the following method. Referring to FIG. 11, a tip column 308 comprising membrane frits 311 and 313 and a packed bed of resin 312 was attached to the output tubing 314.

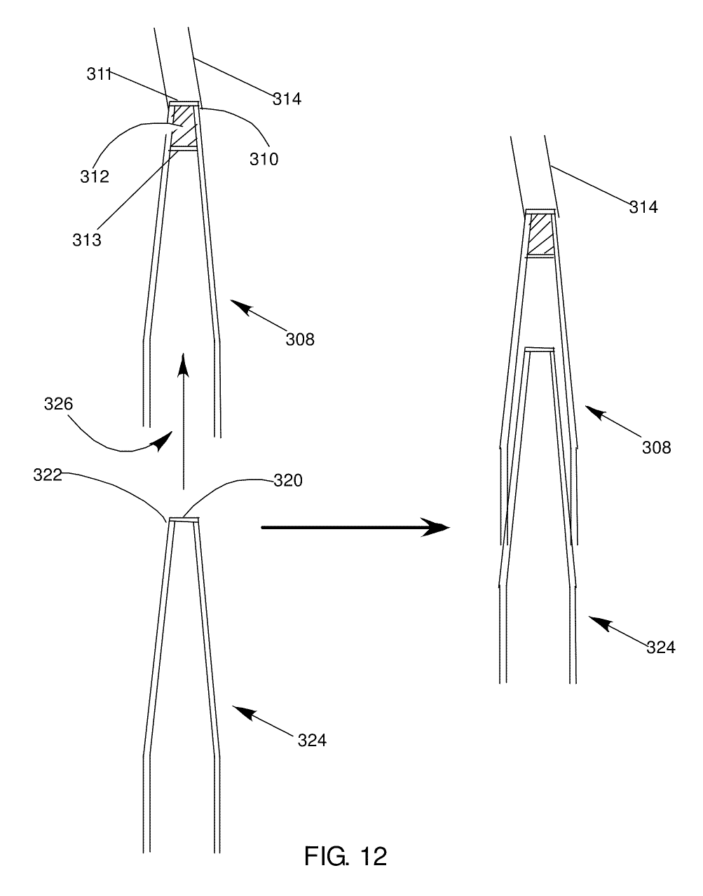

Initially, deionized water is pumped through the bed of media 312 at a constant flow rate, and the baseline backpressure is read off the pressure gauge once the flow and pressure have stabilized, i.e., reached equilibrium. The tip column 308 functions to produce a baseline backpressure when deionized water is pumped through the system. To measure the back pressure of a particular membrane frit, a membrane frit 320 is welded to the narrow end 322 of a pipette tip 324, and the narrow end of the tip 322 is fitted into the wide open end 326 of tip column 308 to form a friction seal (See FIG. 12). The flow and pressure are allowed to stabilize, and the increase in backpressure relative to the baseline backpressure resulting from addition of the membrane is read off the pressure gauge.

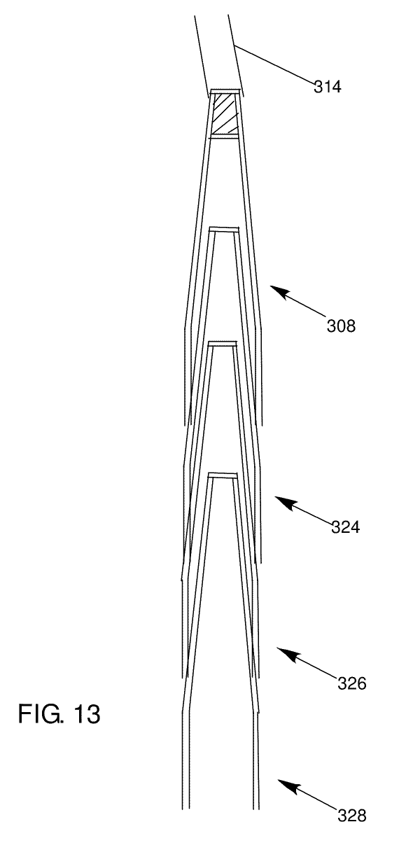

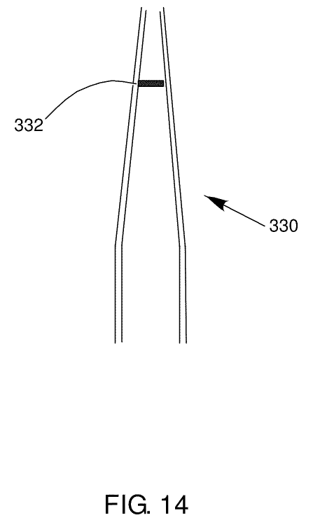

In some experiments, the backpressure was determined for two or more membrane screens attached in series. This was accomplished by friction fitting two or more membrane-tipped pipette tips in series (324, 326 and 328) and attaching to the tip column 308 (see FIG. 13). The increase in backpressure resulting from the plurality of membranes is then read off the gauge once equilibrium has been reached.

In a control experiment, it was determined that attachment of a pipette tip lacking a membrane frit (or several such pipette tips in series) in place of pipette tip 324 did not result in any detectable increase in backpressure. Hence any backpressure detected in the experiments is due solely to the frit or frits.

In one set of experiments, the backpressure for a 1.5 mm diameter 37 micron pore size polyester membrane frit (Spectrum Lab, Cat. No. 146529) was determined at a flow rate of 4 mL/min. The backpressures were determined for different single screens, and it was found that the addition of these membranes resulted in an increase in backpressure of 0.25, 0.3 and 0.3 kPa (1 psi=6.8948 kPa). Two screens were attached in series, and found to result in total increase in an increase in backpressure of 0.4 kPa. Three screens were attached in series, and found to result in an increase in backpressure of 1.1 kPa. Thus, it was concluded that at a flow rate of 4 mL/min, the backpressure of one of these membranes frits is about 0.3 kPa.

In a separate experiment, it was shown that the relationship between backpressure and flow rate is approximately linear. Hence, it can be extrapolated that at a flow rate of 1 mL/min (a typical flow rate when the frits are used in the context of a pipette tip extraction column) the backpressure of these membrane frits is about 0.3/4, or 0.075 kPa.

In another set of experiments, the relation between screen pore size, screen diameter and backpressure was assessed. Polyester membrane frits having pore sizes of 15 micron (Spectrum Lab, Cat. No. 145832), 21 micron (Spectrum Lab, Cat. No. 145833) and 37 micron (Spectrum Lab, Cat. No. 146529) were tested. Two different diameter screens were prepared. The small screen diameter was approximately 0.85 mm and the large screen diameter was 1.4 mm. Because the screens were welded to the tip, the effective diameter varied depending on how much the hot polypropylene flowed from the edge into the screen. This affected the backpressure on the smaller screen diameter much more than the large screen diameter. Three tips each were prepared for each pore size and for each diameter. The results were as follow:

1. Small screen, 15 um, 1 mL/min

### Backpressure: 3.3, 2.7, 1.5 kPa

2. Small screen, 21 um, 4 mL/min
Backpressure: 2.5, 6.3, 3.6 kPa (Therefore effective backpressure at 1 mL/min is extrapolated to be 0.63, 1.6, 0.90 kPa)
3. Small screen, 37 um, 4 mL/min, stack of 3 in series
Backpressure: 2.2 kPa (Therefore effective backpressure of one frit at 1 mL/min is extrapolated to be 0.18 kPa)
4. Large screen, 15 um, 1 mL/min, stack of 3
Backpressure: 6.5 kPa (Therefore effective backpressure at 1 mL/min is extrapolated to be 2.2 kPa)
5. Large screen, 21 um, 4 mL/min, stack of 3 in series
Backpressure: approx. 0.1 kPa (Therefore effective backpressure at 1 mL/min is extrapolated to be 0.0083 kPa)
6. Large screen 37 um, 4 mL/min, stack of 3 in series
Backpressure: approx. 0.05 kPa (Therefore effective backpressure at mL/min 0.0042 is extrapolated to be kPa)

The back pressure was also determined for frits made from porous polymer material, similar to the types of frits used in larger column chromatography. The porous polymer frit were friction fit into pipette tips as shown in FIG. 14 (330 is the pipette tip and 332 is the frit), and the backpressure was determined using the same device and methodology as described above for use with membrane frits. (Note the diameters of the frits reported are cut size. When the frit is pushed into the tip body, the diameter will decrease. Larger diameter frits had to be pushed more firmly into the pipette body to prevent them from dislodging.

All porous polymer frits tested were 1/16 inch thick, and varied in diameter and pore size. The materials tested were a 35 micron pore hydrophilic polymer (3.4 and 4.4 mm diameter) obtained from Scientific Commodities (Lake Havasu City, Az, Cat No. BB2062-35L); a 15-45 micron pore, UHMW Polypropylene polymer obtained from Porex (Cat. No. X-4900) and a 20-25 micron polypropylene polymer obtained from GenPore (Reading, Pa.). The measured backpressures are presented in the following table. The backpressures are substantially higher than those seen with the membrane frits.

| Pore size | Frit diameter | Flow rate | Backpressure |
|---|---|---|---|
| (micron) | (mm) | (mL/min) | (kPa) |
| 35 | 3.4 | 4 | 8.5 |
| 35 | 3.4 | 3 | 6.0 |
| 35 | 3.4 | 2 | 3.6 |
| 35 | 3.4 | 1 | 1.8 |
| 35 | 4.4 | 4 | 4.6 |
| 35 | 4.4 | 3 | 3.5 |
| 35 | 4.4 | 2 | 1.5 |
| 35 | 4.4 | 1 | low |
| 15-45 | 3.4 | 4 | 11.0 |
| 15-45 | 3.4 | 3 | 7.7 |
| 15-45 | 3.4 | 2 | 4.8 |
| 15-45 | 3.4 | 1 | 2.0 |
| 15-45 | 4.4 | 4 | 9.5 |
| 15-45 | 4.4 | 3 | 6.5 |
| 15-45 | 4.4 | 2 | 4.0 |
| 15-45 | 4.4 | 1 | 1.8 |
| 20-25 | 1.4 | 4 | high |
| 20-25 | 1.4 | 3 | 9.0 |
| 20-25 | 1.4 | 2 | 6.0 |
| 20-25 | 1.4 | 1 | 2.5 |

### Example 3

### Desalting a Protein Sample by Size Exclusion

A method and apparatus for desalting a protein sample by size exclusion is depicted in FIG. 15. A desalting tip column is prepared using the methodology provided herein in connection with FIG. 10. The outer column body of the desalting tip is prepared by cutting off the lower end of a 200 μL pipette tip and using this cut off lower section as the outer column body 400 (referring to FIG. 15), corresponding to outer column body 160 of FIG. 10. The total volume of outer column body 400 is about 80 μL, but this is not critical, and in fact a full-size 200 μL pipette tip could be used if so desired. The desalting tip column includes frustoconical ring member 402, upper frit 404 and lower frit 408, corresponding to parts 190, 198 and 174 in FIG. 10. The extraction media chamber 406 is about 40 μL and is packed with a size exclusion media suitable for desalting a protein of interest, e.g., Sephadex G-10, G-15, G-25, G-50 or G-75 (Amersham Biosciences, Piscataway, N.J.). The specific size exclusion media employed will vary depending upon such factors as the size of the protein to be desalted, the nature of constituents of the solution to be desalted, and requirements such as desired speed of the process, yield of product, concentration of product, degree of desalting, etc., as can be determined by one of skill in the art based on the known properties of size exclusion medias such as Sephadex.

The size exclusion resin is hydrated with water, or optionally with a buffer such as PBS. Prior to beginning the actual desalting procedure, air can be blown through the bed of size exclusion media, to drive some or substantially all of the interstitial fluid from the bed. Optionally, the procedure can also be accomplished using a bed that is saturated with solution, e.g., the interstitial spaces are filled with water.

The first step in the desalting procedure is to position a sample to be desalted in a full-size 200 μL pipette tip or pipette-tip based column. Referring again to FIG. 15, pipette tip column 420 is a Ni-NTA extraction tip column containing a 5 μL bed of Ni-NTA resin 412 and a 10 μL drop of elution buffer 414 containing the purified His-tagged protein to be eluted from the column. In other words, this corresponds to the point in the process where the elution buffer has been drawn back and forth through the extraction media for two cycles and is ready to be ejected from the column, along with the purified sample. In the instant example, however, instead of collecting the eluted sample directly, the pipette tip column is inserted into the top end of the desalting tip column and positioned down far enough such that the lower frit 416 of the extraction column is close to the upper frit 404 of the desalting tip column.

The upper end 418 of the extraction tip column is attached to a pipettor, and this pipettor is activated to drive the 10 μL of elution buffer 414 out of the extraction tip and into the bed of size exclusion media (FIG. 15B). The pipettor is then removed, and a chaser pipette tip 422 containing 10 μL of a chaser solution 424 (typically water, or could be a buffer such as PBS) is inserted into the open upper end of the extraction tip column, and positioned such that the lower end 428 of the chaser tip is close to the top of the bed of extraction media 412. The upper end of the chaser tip is attached to a pipettor, and is activated to drive the chaser solution through the bed of extraction media 412, through the bed of size exclusion media 406, and ultimately through the lower frit 408 and out of the column. The eluent, containing the desalted protein, is collected in a collection vial 430.

In an alternative embodiment, the desalting tip column can be made according to the design depicted in FIGS. 1 and 2, according to the methodology accompanying those figures. The bed volume is still 40 uL, but the dimensions of the bed are generally wider and shorter than the bed made according to the method of FIG. 10. An advantage to this alternate tip design is that it does not include the frustoconical ring member 402, which can impede the positioning of the lower frit 416 as close to the upper frit 404 as possible.

In another alternative embodiment of the desalting method, 20 μL of elution buffer is used instead of 10 μL, and no chaser pipette tip or chaser solution is used. Instead, the 20 μL of elution buffer is driven completely through the bed of extraction media 412 and bed of size exclusion media 406, and the desalted sample is collected as described above.

### Example 4

### Automation of the PhyTip Gel Filtration Column

PhyTip gel filtration columns are compatible with use on the PhyNexus MEA Personal Purification System and the Beckman Biomek FX. With some modification, the columns can be made compatible with most 96-channel liquid handling instruments. Four steps are required for use of the PhyTip gel filtration columns for size-based separations. These steps are column equilibration, column conditioning, sample loading and collection of target molecule(s).

PhyTip column equilibration. The PhyTip columns are shipped with glycerol, which acts as a preservative and prevents the media from dehydrating. The glycerol needs to be removed prior to use of the columns. To remove the glycerol, the end of the PhyTip columns are submerged in buffer such as water supplemented with 0.01% sodium azide to act as a preservative. 1 mL of this buffer is added to the top of the columns and these are allowed to equilibrate for at least eight hours overnight. If the glycerol removal step requires faster processing, then the equilibration step can be performed at 42° C. because the glycerol will be less viscous at higher temperatures. Failure to remove the glycerol will result in glycerol contamination in the final, purified sample fractions, or broadening of the target peaks.

PhyTip column conditioning. Once the glycerol has been removed, the PhyTip gel filtration columns are conditioned and the equilibration buffer in the column is exchanged for the final buffer in which the molecule(s) of interest will be collected. The columns are removed from submersion in the equilibration buffer and suspended over a waste collection reservoir and the residual equilibration buffer is allowed to drain out of the column. As the buffer reaches the top frit screen above the resin bed, the fluid flow will stop. Three column volumes of conditioning buffer is added to the top of the PhyTip gel filtration column and the buffer is allowed to drain until all of the buffer has completely entered the resin bed. The flow is generally even but not perfectly so. The flow of liquid stops when the liquid meniscus reaches the frit, then the flow stops. The top frit screen prevents air from entering the resin bed so that column does not dry, crack or channel, which would result in poor performance. The method is paused long enough for all of the columns to reach this state. At this point liquid flow is stopped for all columns until the next aliquot of liquid is added.

PhyTip column sample loading. The PhyTip columns are ready for injection of the sample. The PhyTip columns are transferred to an apparatus that suspends the ends of the columns inside individual collection wells 4 mm above the bottom of the well. Sample is added to the top of the PhyTip column and allowed to enter the resin bed, completely. Every time sample and buffer enters the resin bed, the meniscus of the fluid will stop when it reaches the top frit. The Resin bed will not go dry and the columns are ready for the next buffer addition. The flow through is collected in the well. The table below describes the injection volume range for different PhyTip columns.

Sample collection. Chaser buffer is added to elute the target molecule(s) from the column. The chaser buffer should be the same composition as the conditioning buffer and will be the final desired buffer. The PhyTip columns are moved to a new collection plate and chaser buffer is added to the top of the PhyTip columns. Multiple volumes of the chaser buffer can be added to the columns in a stepwise fashion and each addition can be collected separately to perform fractionation of the samples. This would require moving the columns to a new collection plate prior to the addition of each new chaser fraction. If buffer exchange is the goal, a larger chaser volume is added to the top of the PhyTip column and the target molecule(s) are collected. Care should be taken that the chaser fraction is not too large so as to release the small molecules that are retained in the gel filtration matrix. To efficiently collect the fractions, the PhyTip columns should be suspended an optimal distance above the bottom of the collection well. As the fluid leaves the PhyTip column, it will form a drop attached at the end of the column. The release of the drop is accomplished by having the drop touch the bottom of the well. Once the column is lifted out of the collection plate, the drop will release. The table below shows the suggested chase volumes to be used with different sample volumes and column sizes for buffer exchange and desalting.

Suggested sample and chaser volumes

| Column bed volume (μL) | Sample volume (μL) | Chaser volume (μL) |
|---|---|---|
| 200 | 20 | 150 |
| 200 | 30 | 140 |
| 200 | 40 | 130 |
| 200 | 50 | 120 |
| 200 | 60 | 110 |
| 200 | 70 | 100 |
| 200 | 80 | 90 |
| 200 | 90 | 80 |
| 600 | 100 | 400 |
| 600 | 200 | 300 |
| 600 | 300 | 200 |
| 600 | 400 | 100 |

The steps described above can be fully automated. FIG. 19 shows the MEA setup of PhyTip gel filtration columns for buffer exchange and desalting. The bottom of the page is the front of the unit and the top of the page is the back of the instrument. 144 1 mL transfer tips were placed into Position 1 and rows 1-4 of Position 2 (FIG. 19). Forty-eight 200 μL PhyTip gel filtration columns were placed into Position 2 (FIG. 19, 2). A 95-well plate with 0.5 mL capacity in each well was placed in Position 3 and served as a collection plate (FIG. 19, 3). Position 4 contained a 2 mL deep-well plate with 1 mL of conditioning buffer in rows 1-4 (FIG. 19, 4). Position 7 was affixed with an rack to maintain the rigidity of a 96-well PCR plate, which was placed on top (FIG. 19, 5). Rows 1-4 contained 20-90 μL of samples 1-48 and rows 5-8 contained 20-90 μL (FIG. 19, 5). The MEA added 600 μL of conditioning buffer to the top of 12 PhyTip columns and paused 15 minutes for the conditioning buffer to flow through the columns into waste. The MEA transferred 70 μL samples to the top of the 12-columns and paused 5 minutes for the flow through to collect into waste. The MEA transferred 120 μL of chaser to the top of the 12 columns. The instrument immediately engaged the columns and moved them to row 1 of the collection plate and held them suspended 4 mm above the bottom of the collection well for 10 minutes. This completed the buffer exchange of samples 1-12 and the MEA repeated the process for the next 12 samples until all 48 samples were processed.

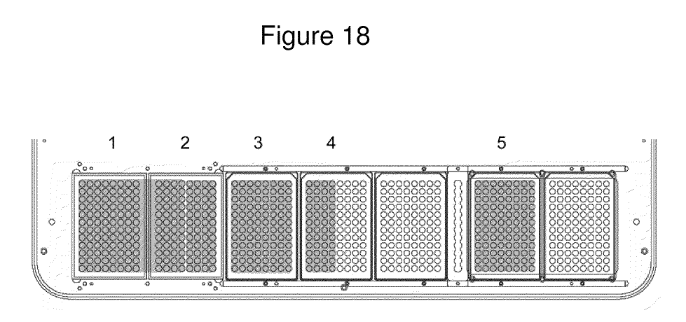

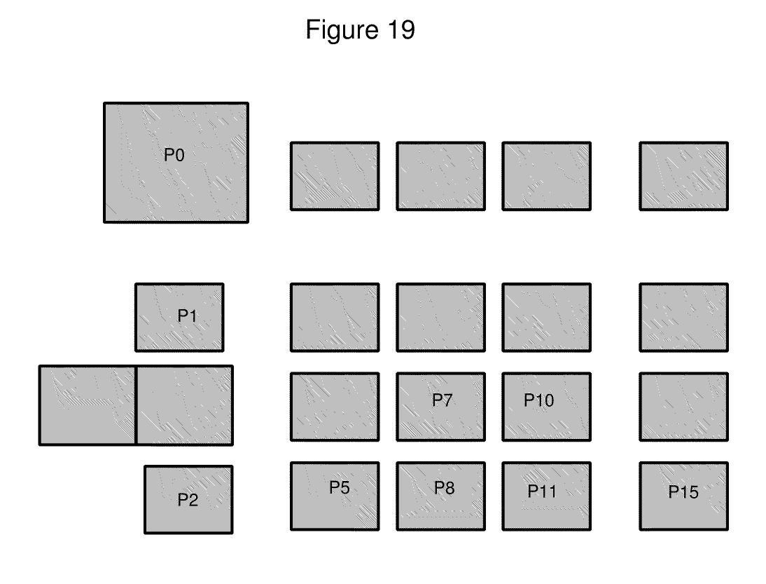

The Beckmam Biomek FX was set up to perform 96 size-based separations using 200 μL PhyTip gel filtration columns. FIG. 18 show how to set up a Beckman Biomek FX for use with PhyTip Gel filtration columns. A box of pipette tips was placed in the Tip Loader (Position P0) and an additional two boxes was placed at Positions (P1) and (P2). The PhyTip columns were placed into a PhyNexus Rack suspended over a waste collection plate in Position (P5). The Rack was made specifically for the Biomeck FX. It was designed to hold 96 PhyTip gel filtration columns, serve as a handle for the Biomek FX gripper function to allow all 96 columns to be moved from one deck position to another, and suspends the PhyTip columns at the proper position above the bottom of the collection well. Position (P1) contained a reservoir plate with 90 mL of Conditioning Buffer. Position (P7) held a 96-well plate containing 96 70 μL Samples. Position (P10) held a 96-well plate containing 120 μL Chaser Buffer in each well. Position (P5) held a 96-well collection plate. The Biomek FX added 600 μL conditioning buffer to the top of the PhyTip columns and the instrument paused for 15 minutes while the conditioning buffer flowed through the resin bed and into the waste collection plate. The instrument next added 70 L sample to each column and the flow through was collected to waste during a 5 minute pause. The instrument moved the columns to the collection plate by employing the gripper function. The instrument added 120 μL chaser to the top of the columns and the flow through was collected.

If fractionation is desired, a stack of Collection Plates are placed in Position (P15). The Biomek FX can take plates from this position and placed them on top of other collection plates at Position (P5). The Rack containing the PhyTip columns can be stacked on top of these empty plates and serve as collection plates for the desired number of samples.

### Example 5

### Separation of Myoglobin Protein from DNP-Glutamate for Desalting

200 μL PhyTip gel filtration columns were equilibrated overnight and conditioned with 700 uL of PBS buffer (10 mM phosphate, 140 mM NaCl, pH 7.4). 20 μL of sample containing brown 2.4 mM myoglobin protein (16,700 MW) and 3.5 mM DNP-glutamate salt (313 MW) was loaded onto PhyTip gel filtration columns. The flow through was collected and the PhyTip columns were chased with 80 μL PBS buffer. The collected fraction was analyzed using a UV spectrometer to calculate protein recovery and salt removal. Myoglobin protein is brown and has a molar extinction coefficient at 409 nm of 2,700M⁻ cm⁻¹. DNP-glutamate is yellow and has a molar extinction coefficient at 364 nm of 487M⁻ cm⁻¹. The concentration of myoglobin and DNP-glutamate was determined using the equation, c=A/εL, where C is the concentration, A is the absorbance, ε is the molar extinction coefficient, and L is the path length.

Myoglobin recovery and salt removal

| | | | Vol. | pmol | pmol DNP- | % myoglobin | % DNP-glutamate |
|---|---|---|---|---|---|---|---|
| | A₃₆₄ | A₄₀₉ | (μL) | myoglobin | glutamate | recovery | removal |
| Myoglobin input | | 1.165 | 20.0 | 47,843.9 | | | |
| Myoglobin sample 1 | | 0.205 | 90.5 | 38,095.5 | | 79.6 | |
| Myoglobin sample 1 | | 0.200 | 94.8 | 38,932.2 | | 81.4 | |
| DNP-glutamate input | 2.440 | | 20.0 | | 70,469.3 | | |
| DNP-glutamate sample 1 | 0.003 | | 88.7 | | 96.1 | | 99.9 |
| DNP-glutamate sample 1 | 0.006 | | 89.3 | | 193.4 | | 99.7 |

### Example 6

### Recovery of Different Proteins and Optimization of PhyTip Gel Filtration Columns

Different molecules have properties, namely shape and molecular weight, which differentiates how they interact with the PhyTip gel filtration column. To determine the appropriate chaser volume to recover a target molecule, it is appropriate to perform a recovery experiment with known standards. 200 μL PhyTip columns were equilibrated and conditioned as in Example 2. 20 μL samples, 3.1 mg/mL final concentration, of human IgG (hIgG, Sigma-Aldrich) spiked into PBS buffer containing 0.05% Tween, was applied to the top of each column. After the sample entered the resin bed, 120 μL PBS buffer was applied to the column to release the hIgG. The sample flow through and chaser was collected and weighed by an analytical scale and measured by HPLC.

IgG recovery

| | Rec. vol. (μL) | A280 | uM | pmoles | % Recovery |
|---|---|---|---|---|---|
| Input | 20.0 | 0.7 | 3.1 | 62.1 | |
| hIgG sample 1 | 133.4 | 0.1 | 0.3 | 45.7 | 73.7 |
| hIgG sample 3 | 110.0 | 0.1 | 0.4 | 41.9 | 67.5 |

### Example 7

### Sample Collection Reproducibility

The efficient collection of the small drops is very important for the performance of the PhyTip gel filtration columns. These small volumes are potentially highly concentrated with the molecule(s) of interest. Procedures were developed to ensure reproducibility in volume recovery. Four PhyTip columns were equilibrated and conditioned as in Example 2. 120 μL PBS was loaded to the top of each column and the flow through was collected. The volume collected was measured by weighing on an analytical scale.

Volume recovery reproducibility

| | Day 1 | Day 2 | Day 3 |
|---|---|---|---|
| PhyTip column # | Rec. vol. (μL) | Rec. vol. (μL) | Rec. vol. (μL) |
| 1 | 122.6 | 118.8 | 133.4 |
| 2 | 132.6 | 106.5 | 121 |
| 3 | 112.6 | 119.4 | 110 |
| 4 | 115.0 | 120.6 | |
| Average | 120.7 | 116.3 | 121.5 |
| Standard Deviation | 9.0 | 6.6 | 11.7 |
| CV | 7.5 | 5.7 | 9.6 |

### Example 8

### PhyTip Column Reproducibility

The PhyTip columns were tested for reproducibility by measuring the recovery of a standard protein spiked into PBS buffer containing 0.05% Tween 20. Twelve 200 μL PhyTip gel filtration columns were equilibrated and conditioned as described in Example 2. 40 μL aliquots of a 2 mg/mL IgG sample were added to the top of the PhyTip columns and the flow through was discarded. The IgG was released by a chaser buffer of 130 μL PBS. The chaser buffer was collected and analyzed by a UV-spectrometer to quantify the sample recovery.

PhyTip gel filtration column performance reproducibility

| | Vol. recovered | [IgG] | mass recovered | % |
|---|---|---|---|---|
| Column # | (uL) | (mg/mL) | (mg) | recovered |
| 1 | 120 | 0.44 | 0.053 | 66 |
| 2 | 125 | 0.54 | 0.068 | 84 |
| 3 | 128 | 0.46 | 0.059 | 74 |
| 4 | 133 | 0.48 | 0.064 | 80 |
| 5 | 130 | 0.43 | 0.056 | 70 |
| 6 | 121 | 0.43 | 0.052 | 65 |
| 7 | 126 | 0.47 | 0.059 | 74 |
| 8 | 119 | 0.53 | 0.063 | 79 |
| 9 | 111 | 0.49 | 0.054 | 68 |
| 10 | 114 | 0.56 | 0.064 | 80 |
| 11 | 98 | 0.61 | 0.060 | 75 |
| 12 | 125 | 0.52 | 0.065 | 81 |
| Ave | 121 | 0.50 | 0.060 | 75 |
| SD | 10 | 0.06 | 0.005 | 6 |
| % CV | 7.9 | 11.3 | 8.5 | 8 |

Performance was enhanced when the pause time between processing the conditioning buffer and addition sample was more carefully controlled. The experiment was repeated and the pause was reduced to 15 minutes from 20 minutes.

Reduce conditioning pause

| | Vol. recovered | [IgG] | Mass recovered | % |
|---|---|---|---|---|
| Column # | (μL) | (mg/mL) | (mg) | recovered |
| 1 | 122 | 0.49 | 0.060 | 75 |
| 2 | 119 | 0.50 | 0.060 | 74 |
| 3 | 122 | 0.50 | 0.061 | 76 |
| 4 | 119 | 0.54 | 0.064 | 80 |
| 5 | 122 | 0.48 | 0.059 | 73 |
| 6 | 123 | 0.51 | 0.063 | 78 |
| Ave | 121 | 0.50 | 0.061 | 76 |
| SD | 2 | 0.02 | 0.002 | 3 |
| % CV | 1.4 | 4.1 | 3.5 | 4 |

### Example 9

### PhyTip Gel Filtration Columns for Use in Size Exclusion Chromatography

PhyTip columns were tested for the ability to separate molecules in a complex sample based upon molecular weight and shape. In some instances, agglomeration was simulated by use of large molecules. PhyTip gel filtration columns were manufactured containing four different types of resin, GE Sephadex S-200, GE Sephadex S-300, ToyoPearl HW-55F, and GE Superose 12 Prep. Samples containing standard proteins of varying molecular weights were used to measure the separation characteristics of each resin. For all experiments, PhyTip columns were made following the standard PhyNexus manufacturing procedure and contained resin beds of 600 μL, 800 μL, or 1000 μL. PhyTip columns were equilibrated and conditioned as per Example 2. 100 μL of sample of varying protein composition was loaded from the top of each PhyTip column and the flow through fraction was collected. Twelve to fourteen 50 μL chaser fractions were collected and analyzed by either UV spectroscopy or HPLC generate a chromatogram.

The standard molecules used in this study are the following:

| Name | Size (MW) |
|---|---|
| Protein X | 350,000 |
| Human immunoglobulin G (hIgG) | 150,000 |
| Bovine serum albumin (BSA) | 67,000 |
| DNPglutamate | 313 |

The high molecular weight Protein X was tested along with the low molecular weight protein, BSA using PhyTip columns containing 600 μL Sephadex S-200. The BSA was releasing early from the column suggesting that the column was either over loaded with BSA or that the BSA was agglomerating. This was determined by comparison with the elution profile of a small molecular weight molecule, DNP-glutamate, which represents a late elution typical of a small molecule. The elution profile of a lower concentration of BSA was tested in addition to PhyTip columns conditioned and chased with different a buffer that promoted denaturation, urea, or with a buffer that contained surfactant, Tween-20.

Detection of molecules after processing by PhyTip
columns containing 600 L GE Sephadex S-200

| | | 5 mg/mL | 0.7 mg/mL | 0.7 mg/mL BSA | | |
|---|---|---|---|---|---|---|
| | Protein X | BSA in | BSA in | in PBS, 0.05% | 3.6 mg/mL | |
| Fraction # | detection | PBS | PBS | Tween-20 | BSA in Urea | DNP-glutamate |
| 1 | | | | | | |
| 2 | | | | | | |
| 3 | | | | | | |
| 4 | | | | | | |
| 5 | | | | | + | |
| 6 | + | + | + | + | + | |
| 7 | + | + | + | + | + | |
| 8 | | + | + | + | + | |
| 9 | | + | | | | |
| 10 | | | | | | |
| 11 | | | | | | + |
| 12 | | | | | | + |
| 13 | | | | | | |
| 14 | | | | | | |

In addition to the Sephadex S-200, three other resins were evaluated for the ability to separate samples containing molecules of different molecular weights.

Detection of molecules after processing by PhyTip
columns containing GE Sephadex S-300

| | 600 μL resin bed volume | | 800 μL resin bed volume | | 1000 μL resin |
|---|---|---|---|---|---|
| | 0.04 mg/mL | 0.7 mg/mL BSA | 0.04 mg/mL | 0.9 mg/mL | bed volume |
| | Protein X in PBS, | in PBS, 0.05% | Protein X | BSA in | 0.8 mg/mL BSA |
| Fraction # | 0.05% Tween-20 | Tween-20 | in PBS | PBS | in PBS |
| 1 | | | | | |
| 2 | | | | | |
| 3 | | | | | |
| 4 | | | | | |
| 5 | | | | | |
| 6 | + | | | + | |
| 7 | + | + | | + | |
| 8 | + | + | + | + | |
| 9 | | + | + | | |
| 10 | | + | | | |
| 11 | | | | | + |
| 12 | | | | | + |
| 13 | | | | | + |
| 14 | | | | | + |

Detection of Protein X after processing by PhyTip
columns containing 600 μL HW-55F or Superose 12

| Fraction # | HW-55F | Superose 12 |
|---|---|---|
| 1 | | |
| 2 | | |
| 3 | | |
| 4 | | |
| 5 | | |
| 6 | + | |
| 7 | + | + |
| 8 | + | + |
| 9 | | + |
| 10 | | |
| 11 | | |
| 12 | | |
| 13 | | |
| 14 | | |

### Example 10

### PhyTip Columns for Separation of Nucleic Acid Monomers from Oligonucleotides

Nucleic acids including but not limited to DNA, RNA, DNA/RNA hybrids and nucleic acids containing nucleotide analogs and modifications will be purified of free nucleotides, free labels, salts and other small molecules by PhyTip gel filtration columns. Additionally, buffer exchange is often required for enzymatic reaction compatibility. Oligonucleotides of different composition and different lengths will be mixed with a small fluorescent dye. These samples will be processed by 600 μL PhyTip gel filtration columns equilibrated in PBS buffer. 100 μL samples will be applied to the columns and the flow through will be collected. 100 μL of PBS will be applied to the top of the column and the flow through will be collected in a separate, clean tube. This fractionation will continue for seven more fractions of 100 μL PBS. Sample fractions will be analyzed by UV spectroscopy and the nucleic acid recovery will be measured by absorbance at 260 nm. The contaminating dye will be measured at the appropriate absorbance and the conditions for best nucleic acid recovery and dye removal will be determined.

### Example 11

### Obtaining Flow and Performance Consistency from PhyTip Columns

The construction of PhyNexus gel-filtration columns is critical to the flow rate. If the resin is over packed then flow rates will be slowed considerably. If there is a gap between the top frit and the resin bed then an air bubble will be trapped when fluid is introduced to the top of the column and no flow will occur.

A set of columns must contain the same volume of resin to flow consistently. Several salts were tested to raise the density of the resin slurry to maintain a consistent suspension. The control slurry consisted of 2 g Sephadex G25 resin brought up to 20 mL with a 0.01% sodium azide solution. Another identical slurry was made except it was supplemented with 24 g cesium chloride. The addition of cesium chloride resulted in slurry staying in suspension with less agitation. 24 gel-filtration columns were packed with 200 μL of each resin and washed with 6 mL of 0.01% sodium azide. The flow characteristics of these packed bed columns was measured before the top frits were placed above the resin bed. 700 μL 0.01% sodium azide was added to the top of each column and the time for the fluid to completely enter the resin bed was recorded (Table 1). This experiment was done in triplicate. The results of this showed that columns manufactured with cesium chloride flowed slightly slower (11 minutes, 38 seconds on average) than those made without (9 minutes 50 seconds on average).

The impact of the top frit was tested by taking the columns manufactured described above and adding the top screen at various heights. First the 24 PhyTip columns manufactured with cesium chloride had top frits inserted to where the top frit was just touching the resin bed. Slight compression of the resin bed may have occurred but it was minimal (<1 mm). Again, 700 μL of 0.1% sodium azide was added to the top of the columns and the time for fluid to completely flow through the resin bed was recorded (Table 2). This experiment was run in triplicate. Mean flow times for these columns was 12 minutes, 0 seconds, which was slightly longer than the columns without inserts. Columns #9 and #17 had a slight gap between the top of the resin bed and the top frit. This was noticed after the first trial, which is why they did not flow. The top frits were re-seated prior to the next run by having the frit just touch the resin. The data from these two columns was not included in the mean flow time calculation. To test how compression of the top screen affects flow, these columns were stressed by pushing the top frit down approximately 1 mm. Four measurements for the time for 700 μL of 0.1% sodium azide to completely flow through the resin bed was recorded (Table 2). The average flow time for these column was 15 minutes and 13 seconds. The impact of compressing the top frit an additional 1 mm resulted in slowing the processing time to 21 minutes and 45 seconds (Table 3).

To test hot a gap affects the flow of fluid through the resin bed, 24 columns that were manufactured without CsCl, described above, were used to test inserts of either 1.5 mm above the resin bed or with less than 1 mm of compression (Table 4). The result of a less than 1 mm compression resulted in a flow processing time of 11 minutes, 31 seconds.

A final variation of the top screen was tested to attempt to alleviate the compression of the resin bed. Columns 9-16 manufactured without CsCl was used to frit screens with a slit cut through the diameter. When these frits were placed 1.5 mm above the resin bed, there is no flow (Table 5). When the frits were re-seated to compress the resin bed by <1 mm, then the mean flow was 11 minutes, 52 seconds. Then the compression increased to 1 mm, the flow was prolonged to 1 2 minutes, 28 seconds.

TABLE 1
PhyTip columns manufactured with and without cesium chloride in the resin slurry

| | Slurry composition: 0.01% sodium azide | | | | Slurry composition: 0.01% sodium azide, CsCl | | | |
|---|---|---|---|---|---|---|---|---|
| | Time to | Time to | Time to | | Time to | Time to | Time to | |
| | process | process | process | Ave. | process | process | process | Ave. |
| PhyTip | 700 μL - 1 | 700 μL - 2 | 700 μL - 3 | processing | 700 μL - 1 | 700 μL - 2 | 700 μL - 3 | processing |
| column # | (min.) | (min.) | (min.) | time (min.) | (min.) | (min.) | (min.) | time (min.) |
| 1 | 8.75 | 8.75 | 9.00 | 8.83 | 10.00 | 10.25 | 10.50 | 10.25 |
| 2 | 11.50 | 10.75 | 10.50 | 10.92 | 10.75 | 10.75 | 10.50 | 10.67 |
| 3 | 10.25 | 10.25 | 10.25 | 10.25 | 12.00 | 12.00 | 12.25 | 12.08 |
| 4 | 9.75 | 9.25 | 8.75 | 9.25 | 11.00 | 10.75 | 11.00 | 10.92 |
| 5 | 9.75 | 9.25 | 9.25 | 9.42 | 12.00 | 12.00 | 12.25 | 12.08 |
| 6 | 9.75 | 9.25 | 10.25 | 9.75 | 10.25 | 10.25 | 10.25 | 10.25 |
| 7 | 10.25 | 9.75 | 9.75 | 9.92 | 10.25 | 10.25 | 11.50 | 10.67 |
| 8 | 9.25 | 9.75 | 9.75 | 9.58 | 11.00 | 10.75 | 11.50 | 11.08 |
| 9 | 9.25 | 10.00 | 9.00 | 9.42 | 12.50 | 13.00 | 13.00 | 12.83 |
| 10 | 9.75 | 10.50 | 9.50 | 9.92 | 11.00 | 11.50 | 11.50 | 11.33 |
| 11 | 10.25 | 10.50 | 9.50 | 10.08 | 11.00 | 11.50 | 11.50 | 11.33 |
| 12 | 9.75 | 10.00 | 9.75 | 9.83 | 11.00 | 11.50 | 11.50 | 11.33 |
| 13 | 10.25 | 10.50 | 9.75 | 10.17 | 12.25 | 12.25 | 12.50 | 12.33 |
| 14 | 10.50 | 10.50 | 10.25 | 10.42 | 12.50 | 13.00 | 13.25 | 12.92 |
| 15 | 9.50 | 9.25 | 9.50 | 9.42 | 11.50 | 12.25 | 12.25 | 12.00 |
| 16 | 9.25 | 9.75 | 10.25 | 9.75 | 11.50 | 12.25 | 12.25 | 12.00 |
| 17 | 8.50 | 9.00 | 9.25 | 8.92 | 10.25 | 10.00 | 10.75 | 10.33 |
| 18 | 10.00 | 10.25 | 10.00 | 10.08 | 11.50 | 11.25 | 11.25 | 11.33 |
| 19 | 10.00 | 10.00 | 10.25 | 10.08 | 11.50 | 13.00 | 12.75 | 12.42 |
| 20 | 10.00 | 10.00 | 10.25 | 10.08 | 11.50 | 12.50 | 12.75 | 12.25 |
| 21 | 9.50 | 10.25 | 9.75 | 9.83 | 11.50 | 11.75 | 11.75 | 11.67 |
| 22 | 10.25 | 10.25 | 9.75 | 10.08 | 10.50 | 11.50 | 10.75 | 10.92 |
| 23 | 10.25 | 10.00 | 9.75 | 10.00 | 11.50 | 13.25 | 12.75 | 12.50 |
| 24 | 9.50 | 10.00 | 9.75 | 9.75 | 10.50 | 11.25 | 11.75 | 11.17 |
| Ave. | 9.82 | 9.91 | 9.74 | 9.82 | 11.22 | 11.61 | 11.75 | 11.53 |

TABLE 2
PhyTip columns manufactured with top frit insert screens

| | No compression of resin bed | | | | 1 mm compression of resin bed | | | | |
|---|---|---|---|---|---|---|---|---|---|
| | Time to | Time to | Time to | Ave. | Time to | Time to | Time to | Time to | Ave. |
| | process | process | process | processing | process | process | process | process | processing |
| PhyTip | 700 μL - 1 | 700 μL - 2 | 700 μL - 3 | time | 700 μL - 1 | 700 μL - 2 | 700 μL - 3 | 700 μL - 4 | time |
| column # | (min.) | (min.) | (min.) | (min.) | (min.) | (min.) | (min.) | (min.) | (min.) |
| 1 | 10.50 | 12.25 | 11.75 | 11.50 | 12.25 | 12.50 | 13.50 | 13.25 | 12.88 |
| 2 | 9.50 | 10.50 | 11.75 | 10.58 | 14.50 | 15.00 | 14.75 | 15.00 | 14.81 |
| 3 | 12.00 | 13.25 | 13.75 | 13.00 | 13.00 | 14.00 | 13.50 | 13.75 | 13.56 |
| 4 | 10.25 | 11.50 | 11.75 | 11.17 | 14.00 | 14.25 | 14.75 | 15.00 | 14.50 |
| 5 | 11.00 | 12.75 | 13.25 | 12.33 | 14.00 | 15.00 | 14.75 | 14.75 | 14.63 |
| 6 | 10.00 | 12.25 | 11.75 | 11.33 | 12.75 | 13.50 | 13.25 | 14.00 | 13.38 |
| 7 | 10.00 | 11.00 | 11.75 | 10.92 | 13.75 | 15.00 | 14.75 | 14.75 | 14.56 |
| 8 | 10.00 | 11.00 | 10.75 | 10.58 | 15.50 | 15.50 | 16.50 | 16.75 | 16.06 |
| 9 | No Flow | 13.75 | 14.00 | 13.88 | 13.25 | 14.25 | 13.50 | 14.50 | 13.88 |
| 10 | 11.50 | 12.00 | 12.25 | 11.92 | 13.25 | 14.25 | 13.50 | 14.00 | 13.75 |
| 11 | 11.50 | 12.00 | 12.25 | 11.92 | 17.50 | 18.25 | 18.25 | 18.50 | 18.13 |
| 12 | 11.50 | 12.00 | 12.25 | 11.92 | 17.00 | 17.50 | 14.25 | 14.00 | 15.69 |
| 13 | 12.00 | 12.00 | 12.25 | 12.08 | 15.25 | 15.75 | 16.00 | 15.75 | 15.69 |
| 14 | 12.50 | 12.75 | 13.50 | 12.92 | 12.50 | 13.25 | 14.00 | 14.50 | 13.56 |
| 15 | 10.25 | 10.25 | 11.00 | 10.50 | 14.50 | 16.00 | 16.25 | 16.25 | 15.75 |
| 16 | 10.25 | 10.25 | 11.00 | 10.50 | 14.75 | 14.25 | 14.25 | 14.25 | 14.38 |
| 17 | No Flow | 13.50 | 15.50 | 14.50 | 12.25 | 13.75 | 12.75 | 13.50 | 13.06 |
| 18 | 11.00 | 11.50 | 12.00 | 11.50 | 17.50 | 17.75 | 18.25 | 18.25 | 17.94 |
| 19 | 12.00 | 12.50 | 12.50 | 12.33 | 14.00 | 14.75 | 15.25 | 15.25 | 14.81 |
| 20 | 17.00 | 15.75 | 14.75 | 15.83 | 15.75 | 16.50 | 17.00 | 16.50 | 16.44 |
| 21 | 11.00 | 11.75 | 11.30 | 11.35 | 17.25 | 18.75 | 18.25 | 18.50 | 18.19 |
| 22 | 9.50 | 11.50 | 12.00 | 11.00 | 13.50 | 14.75 | 15.25 | 16.50 | 15.00 |
| 23 | 12.25 | 12.75 | 13.75 | 12.92 | 16.00 | 18.50 | 17.50 | 18.00 | 17.50 |
| 24 | 11.75 | 11.25 | 11.25 | 11.42 | 16.00 | 17.25 | 17.50 | 17.00 | 16.94 |
| Ave. | 11.24 | 12.08 | 12.42 | 12.00 | 14.58 | 15.43 | 15.31 | 15.52 | 15.21 |

TABLE 3
Compressing the resin bed by 2 mm

| PhyTip | Time to process | Time to process | Ave. processing |
|---|---|---|---|
| column # | 700 μl −1 (min.) | 700 μL −2 (min.) | time (min.) |
| 1 | 19.00 | 18.25 | 18.63 |
| 2 | 17.00 | 15.75 | 16.38 |
| 3 | 14.00 | 14.00 | 14.00 |
| 4 | 25.00 | 24.75 | 24.88 |
| 5 | 21.00 | 20.00 | 20.50 |
| 6 | 23.75 | 23.50 | 23.63 |
| 7 | 25.00 | 23.50 | 24.25 |
| 8 | 25.00 | 23.75 | 24.38 |
| 9 | 19.25 | 19.75 | 19.50 |
| 10 | 20.00 | 19.75 | 19.88 |
| 11 | 23.25 | 24.00 | 23.63 |
| 12 | 20.50 | 20.50 | 20.50 |
| 13 | 26.00 | 26.00 | 26.00 |
| 14 | 17.25 | 16.50 | 16.88 |
| 15 | 27.00 | 26.50 | 26.75 |
| 16 | 27.00 | 26.50 | 26.75 |
| 17 | 21.25 | 20.25 | 20.75 |
| 18 | 28.00 | 28.00 | 28.00 |
| 19 | 24.00 | 21.50 | 22.75 |
| 20 | 22.50 | 21.50 | 22.00 |
| 21 | 23.50 | 21.75 | 22.63 |
| 22 | 19.50 | 18.50 | 19.00 |
| 23 | 18.00 | 17.00 | 17.50 |
| 24 | 24.00 | 21.75 | 22.88 |
| Ave. | | | |

TABLE 4
Minimal compression of the resin bed

| | 1.5 mm gap | | | |
|---|---|---|---|---|
| | between resin | | | |
| | bed and frit | Compression of resin bed by <1 mm | | |
| | Time to | Time to | Time to | |
| | process | process | process | |
| PhyTip | 700 μL | 700 μL | 700 μL | Ave. processing |
| column # | −1 (min.) | −1 (min.) | −2 (min.) | time (min.) |
| 1 | No Flow | 11.75 | 12.25 | 12.00 |
| 2 | No Flow | 12.75 | 14.00 | 13.38 |
| 3 | No Flow | 12.00 | 12.50 | 12.25 |
| 4 | No Flow | 14.50 | 14.75 | 14.63 |
| 5 | No Flow | 12.00 | 12.25 | 12.13 |
| 6 | No Flow | 14.00 | 13.50 | 13.75 |
| 7 | No Flow | 11.75 | 11.75 | 11.75 |
| 8 | No Flow | 11.75 | 12.25 | 12.00 |
| Ave. | | 12.56 | 12.91 | 12.73 |

TABLE 5
Frit with a slit through the diameter of the screen

| | 1.5 mm | | | | |
|---|---|---|---|---|---|
| | gap | | | | |
| | between | | | | Compression |
| | resin bed | Compression of resin | | | of resin bed |
| | and frit | bed by <1 mm | | | by 1 mm |
| | Time to | Time to | Time to | Ave. | Time to |
| | process | process | process | processing | process |
| PhyTip | 700 μL - 1 | 700 μL - 1 | 700 μL - 2 | time | 700 μL - 1 |
| column # | (min.) | (min.) | (min.) | (min.) | (min.) |
| 9 | No Flow | 10.75 | 11.25 | 11.00 | 10.75 |
| 10 | No Flow | 11.75 | 11.50 | 11.63 | 11.00 |
| 11 | No Flow | 11.50 | 12.50 | 12.00 | 12.25 |
| 12 | No Flow | 10.50 | 11.25 | 10.88 | 12.25 |
| 13 | No Flow | 12.00 | 11.75 | 11.88 | 12.25 |
| 14 | No Flow | 11.75 | 11.50 | 11.63 | 12.25 |
| 15 | No Flow | 12.00 | 11.75 | 11.88 | 16.50 |
| 16 | No Flow | 10.75 | 11.75 | 11.25 | 12.50 |
| Ave. | | 11.38 | 11.66 | 11.52 | 12.47 |

While the invention has been described in connection with specific embodiments thereof, it will be understood that it is capable of further modifications and this application is intended to cover and variations, uses, or adaptations of the invention that follow, in general, the principles of the invention, including such departures from the present disclosure as come within known or customary practice within the art to which the invention pertains and as may be applied to the essential features hereinbefore set forth. Moreover, the fact that certain aspects of the invention are pointed out as preferred embodiments is not intended to in any way limit the invention to such preferred embodiments.

What is claimed is:

1. A parallel method for purifying an analyte from a sample solution using gel filtration comprising the steps of:
a. providing a plurality of gel filtration pipette tip columns, wherein each pipette tip column is comprised of
i) a column body having an open upper end, an open lower end, and an open channel between the upper and lower end of the column body, wherein the column body is comprised of a modified pipette tip;
ii) a bottom frit bonded to and extending across the open channel, wherein the bottom frit is located at the open lower end of the column body;
iii) a packed bed of gel filtration media positioned above the bottom frit,
b. passing a conditioning solution through the columns by gravity flow;
c. passing a sample solution containing an analyte through the columns by gravity flow;
d. passing a chaser solution through the columns by gravity flow; and
e. collecting the purified analyte, wherein the volume of purified analyte obtained from the columns has a coefficient of variation of less than 10.
